SEQUENCE LISTING (1 sequence)

SEQ ID NO: 1 - RNA, 22 nt, synthetic construct
  source 1..22: mol_type = other RNA
aacagcacaaactactacctca

(19) United States
(12) Patent Application Publication
(10) Pub. No.: US 2025/0270557 A1
(43) Pub. Date: Aug. 28, 2025

(54) COMPOSITION AND METHOD FOR TREATING LUNG DISEASES

(71) Applicant: Taipei Medical University, Taipei City (TW)

(72) Inventors: Chun-Jen HUANG, New Taipei City (TW); Chao-Yuan CHANG, New Taipei City (TW)

(21) Appl. No.: 19/060,464

(22) Filed: Feb. 21, 2025

Related U.S. Application Data
(60) Provisional application No. 63/557,089, filed on Feb. 23, 2024.

(30) Foreign Application Priority Data
Nov. 8, 2024 (TW) 113143017

(51) Int. Cl. C12N 15/113; A61K 9/50; A61P 11/00

(52) U.S. Cl. CPC C12N 15/113; A61K 9/5068; A61P 11/00; C12N 2310/141; C12N 2310/3513

(57) ABSTRACT

Provided is a composition including an extracellular vesicle engineered to carry a payload. Also provided is a method for preventing or treating a lung disease by administering the composition.

### CROSS REFERENCE TO RELATED APPLICATIONS

This patent application claims the benefit of U.S. Provisional Ser. No. 63/557,089, filed Feb. 23, 2024, the entire contents of which are incorporated herein by reference.

### REFERENCE TO AN ELECTRONIC SEQUENCE LISTING

The content of the electronic sequence listing (File name: TPMU-0020US_Sequence Listing.xml; Size: 2 kilobytes; and Date of Creation: Feb. 19, 2025) is herein incorporated by reference in its entirety.

### BACKGROUND

### Technical Field

The present disclosure provides compositions and methods for treating lung diseases, e.g., acute respiratory distress syndrome (ARDS) and acute lung injury. The composition includes an extracellular vesicle (EV) loaded with at least a microRNA (miRNA) and a pharmaceutically acceptable excipient thereof.

### Description of Related Art

Acute respiratory distress syndrome (ARDS) is an acute, diffuse, inflammatory form of lung injury and is usually life-threatening. Associated with capillary endothelial injury and diffuse alveolar damage, ARDS is characterized by poor oxygenation, pulmonary infiltrates, and acute onset. Therefore, patients present dyspnea and hypoxemia, progressively worsen within 6 to 72 hours of the inciting event, and frequently require mechanical ventilation and intensive care unit-level care.

ARDS carries a high mortality, and few effective therapeutic modalities exist to combat this condition. Currently, the main treatment strategy is supportive care and focuses on reducing shunt fraction, increasing oxygen delivery, decreasing oxygen consumption, and avoiding further injury. Patients are mechanically ventilated, guarded against fluid overload with diuretics, and given nutritional support until improvement is observed.

Hence, there remains an unmet need in a medication or method that provides efficient and effective prevention or treatment of lung diseases.

### SUMMARY

The present disclosure provides a pharmaceutical composition comprising an extracellular vesicle engineered to be loaded with a let-7i-5p miRNA from the lethal 7 gene, e.g., Homo sapiens (hsa)-let-7i-5p miRNA, and a pharmaceutically acceptable excipient thereof.

In at least one embodiment of the present disclosure, the extracellular vesicle undergoes electroporation, lipofection, sonication, or contact with calcium chloride to load the miRNA.

In at least one embodiment of the present disclosure, the extracellular vesicle is derived from an animal cell. In at least one embodiment of the present disclosure, the animal cell is a mammalian cell. In at least one embodiment of the present disclosure, the mammalian cell is a non-stem cell, a mesenchymal stem cell, or an immune cell. In at least one embodiment of the present disclosure, the immune cell is a macrophage.

In at least one embodiment of the present disclosure, the extracellular vesicle is derived from a cell grown in vitro or a body fluid of a subject. In at least one embodiment of the present disclosure, the extracellular vesicle is an exosome.

Also provided by the present disclosure is a method for preventing or treating a lung disease in a subject in need thereof. In at least one embodiment, the method of the present disclosure comprises administering the pharmaceutical composition as described above to the subject in need thereof.

In at least one embodiment of the present disclosure, provided is a method for preventing or treating acute respiratory distress syndrome in a subject in need thereof. In at least one embodiment, the method of the present disclosure comprises administering to the subject an effective amount of the above pharmaceutical composition. In at least one embodiment of the present disclosure, the acute respiratory distress syndrome is induced by sepsis. In another embodiment of the present disclosure, the acute respiratory distress syndrome is induced by aspiration pneumonia.

In at least one embodiment of the present disclosure, the administration reduces lung injury. In at least one embodiment of the present disclosure, the administration reduces at least one of inflammation, oxidation, cell death, and apoptosis of lung tissues. In at least one embodiment of the present disclosure, the administration improves lung function, including increasing inspiratory capacity of lung, increasing dynamic compliance of lung, increasing forced expiratory volume of lung, lowering airway resistance, and lowering airway elastance.

### BRIEF DESCRIPTION OF DRAWINGS

The present disclosure can be more understood by reading the following descriptions of the embodiments, with reference made to one or more of the accompanying drawings below.

FIGS. 1A to 1D show the characteristics and cargo of engineered exosomes (EExo). FIG. 1A shows the images presenting the morphology of engineered exosomes. FIG. 1B shows the particle sizes and numbers of engineered exosomes. FIG. 1C is the immunoblot analysis result showing the expression of exosomal surface markers CD63 and CD9 in engineered exosomes. FIG. 1D shows the next-generation sequencing (NGS) analysis results on the content of hsa-let-7i-5 p miRNA in exosomes. Scale bar: 200 nm. Vector: exosomes isolated from RAW264.7 cells. EExo: engineered exosomes isolated from RAW264.7 cells with plasmid-mediated hsa-let-7i-5p miRNA overexpression. PBS: phosphate-buffered saline.

FIGS. 2A to 2D show the characteristics of isolated exosomes from genetically modified RAW264.7 cells and human placenta mesenchymal stem cells (hpMSC). FIG. 2A shows the representative transmission electron microscopic images of isolated exosomes. FIG. 2B shows the representative gel photography of markers ALIX and CD9 in isolated exosomes using the Simple Western method. FIG. 2C shows the results of particle sizing assay of isolated exosomes. FIG. 2D shows the microRNA concentrations of hsa-let-7i-5p in isolated exosomes. Data were obtained from 3 exosome batches in each group and presented as mean±standard deviations. *p<0.05, versus the RExo group. EExo: engineered exosomes from RAW264.7 cells overexpressing hsa-let-7i-5p. MExo: exosomes from human placenta-derived mesenchymal stem cells. RExo: exosomes from RAW264.7 cells without genetic modification.

FIGS. 3A to 3C show the results of biodistribution assay and pharmacokinetics of engineered exosomes loaded with hsa-let-7i-5p miRNA by intraperitoneal administration. FIGS. 3A and 3B show in vivo biodistribution of exosomes conjugated with Cy7 mono-NHS ester (1×10⁹ particles per mouse) in the heart, lung, liver, kidney, spleen, and bladder of the mice, using ex vivo bioluminescence imaging assay, measured at 0, 2, 24, and 48 h after intraperitoneal administration. FIG. 3C shows the result of pharmacokinetic analysis of engineered exosomes, where the plasma concentrations of engineered exosomes were measured through the assay of ExoCounter.

FIGS. 4A to 4C show the results of biodistribution assay and pharmacokinetics of engineered exosomes loaded with hsa-let-7i-5p miRNA by intratracheal administration. FIGS. 4A and 4B show in vivo biodistribution of exosomes conjugated with Cy7 mono-NHS ester (1×10⁹ particles per mouse) in the heart, lung, liver, kidney, spleen, and bladder of the mice, using ex vivo bioluminescence imaging assay, measured at 0, 2, 24, and 48 h after intratracheal administration. FIG. 4C shows the result of pharmacokinetic analysis of engineered exosomes, where the plasma concentrations of engineered exosomes were measured through the assay of ExoCounter.

FIGS. 5A to 5C show the results of biodistribution assay and pharmacokinetics of isolated exosomes from genetically modified RAW264.7 cells and human placenta mesenchymal stem cells (hpMSC). FIGS. 5A and 5B show, respectively, the biodistribution of hpMSC exosomes (MExo, 1×10⁸ particles per mouse) and engineered exosomes (EExo, 1×10⁹ particles per mouse) conjugated with Cy7 mono NHS ester in the heart, lung, liver, kidney, spleen, and bladder of the mice, using ex vivo bioluminescence imaging assay, measured at 0, 2, 24, and 48 hours after intraperitoneal administration. Data were obtained from 3 mice sacrificed at each time point in both groups. FIG. 5C shows the pharmacokinetic analysis of MExo and EExo. The plasma concentrations of MExo and EExo were measured through the assay of Cy7 mono NHS ester signal intensities. The MExo and EExo concentrations were measured at 0.5 h after intraperitoneal administration and were used as the baseline. Data were obtained from 3 mice in each group. hpMSC exosomes (MExo): exosomes from human placenta-derived mesenchymal stem cells. Engineered exosomes (EExo): engineered exosomes from RAW264.7 cells overexpressing hsa-let-7i-5p.

FIGS. 6A to 6M show the effects of intraperitoneal administration of engineered exosomes loaded with hsa-let-7i-5p miRNA on alleviating sepsis-induced lung injury. FIG. 6A shows the 48-hour survival rate of each mouse group, where * denotes p<0.05 as the LPS group vs. the Sham group and #denotes p<0.05 between the Sham/EExo group vs. the LPS group and also the LPS group vs. the LPSEExo group. FIG. 6B shows the histological characteristics evaluation with hematoxylin and eosin (HE) stain and lung injury scores. FIG. 6C shows the wet/dry weight (W/D) ratio. FIGS. 6D to 6I show the results on assessment of lung function, including inspiratory capacity, dynamic compliance, resistance, elastance (Ers), and forced expiratory volume. FIGS. 6J to 6M show the cell composition of bronchoalveolar fluids (BALFs). Sham: the sham operation group where mice were administered with normal saline only. EExo: the engineered exosomes (1×10⁹ particles per mouse) group where mice were administered with engineered exosomes. LPS: the sepsis model group where sepsis was induced in mice by lipopolysaccharide (25 mg/kg, intraperitoneally). LPSEExo: the LPS plus EExo group where the sepsis-induced mice were administered with engineered exosomes.

FIGS. 7A to 7I show the effects of intratracheal administration of engineered exosomes loaded with hsa-let-7i-5p miRNA on alleviating aspiration pneumonia-induced lung injury. FIG. 7A shows the histological characteristics evaluation with hematoxylin and eosin (HE) stain. FIG. 7B shows the wet/dry weight (W/D) ratio. FIGS. 7C to 7E show the results on assessment of lung function, including inspiratory capacity, dynamic compliance, and resistance. FIGS. 7F to 7I show the cell composition of bronchoalveolar fluids (BALFs). Sham: the sham operation group where mice were administered with normal saline only. EExo: the engineered exosomes (1×10⁹ particles per mouse) group where mice were administered with engineered exosomes. AP: the aspiration pneumonia model group where aspiration pneumonia was induced in mice by gastric content. APEExo: the AP plus EExo group where aspiration pneumonia-induced mice were administered with engineered exosomes.

FIG. 8 shows the 48-hour (48-h) survival rates of mice in a preliminary test on dose of exosomes required for survivorship assay in LPS-treated mice. Sham: the normal saline group. EExo group: the normal saline plus engineered exosomes group. LPS: the lipopolysaccharide (LPS)-treated group. LEExo(1) and LEExo(2): the LPS-treated group plus 1 dosage engineered exosomes (1) or 2 dosage engineered exosomes (2). LEExoi: the LPS-treated group plus inhibitor-treated engineered exosomes group.

FIGS. 9A and 9B show the survivorship and plasma cytokines of the LPS-induced monomicrobial sepsis animal model. FIG. 9A shows the 48-hour (48-h) survival rates, as determined by calculating the number of mice survived the 48-hour observational duration in each group after normal saline or lipopolysaccharide administration. Data were derived from 6 mice in the Sham, MExo, and EExo groups and 12 mice from the LPS, LMExo, LMExoi, LEExo, and LEExoi groups. *p<0.05, the LPS group versus the Sham group. #p<0.05, the LEExo group versus the LPS group. ∧p<0.05, the LEExoi group versus the LEExo group. FIG. 9B shows plasma concentrations of tumor necrosis factor-α (TNF-α), interleukin-1β (IL-1β), and IL-6, measured using enzyme-linked immunosorbent assay. Data were obtained from five mice in each group. All assays were measured at 48 hours after normal saline or lipopolysaccharide administration. Data represented as mean +standard deviations. *p<0.05, versus the Sham group. #p<0.05, versus the LPS group. †p<0.05, the LMExoi group versus the LMExo group. ∧p<0.05, the LEExoi group versus the LEExo group. Sham: the normal saline group. MExo group: the normal saline plus hpMSC exosomes group. EExo group: the normal saline plus engineered exosomes group. LPS: the lipopolysaccharide-treated (LPS) group. LMExo: the LPS-treated plus hpMSC exosomes group. LMExoi: the LPS-treated plus inhibitor-treated hpMSC exosomes group. LEExo: the LPS-treated plus engineered exosomes group. LEExoi: the LPS-treated plus inhibitor-treated treated engineered exosomes group.

FIGS. 10A to 10D show lung injury and function assessment in the LPS-induced monomicrobial sepsis animal model. FIG. 10A shows representative histological characteristics of lung injury in lung tissues stained with hematoxylin and eosin, evaluated using a light microscope (200×) and the data of lung injury scores. Data were obtained from 6 mice in each group. FIG. 10B shows the wet/dry weight ratio (W/D ratio) of the lung tissues. Data were obtained from 6 mice in each group. FIG. 10C shows the cell number of white blood cells (WBC), neutrophils, lymphocytes, and monocytes in collected bronchoalveolar lavage fluid (BALF). Data were obtained from 6 mice in each group. All assays were measured at 48 hours after normal saline or lipopolysaccharide administration. Data represented as mean±standard deviations. *p<0.05, versus the Sham group. #p<0.05, versus the LPS group. †p<0.05, the LMExoi group versus the LMExo group. ∧p<0.05, the LEExoi group versus the LEExo group. FIG. 10D shows assessment of lung functions represented by inspiratory capacity, airway resistance, and dynamic compliance. Data were obtained from 6 mice from each group for each parameter. Sham: the normal saline group. MExo group: the normal saline plus hpMSC exosomes group. EExo group: the normal saline plus engineered exosomes group. LPS: the lipopolysaccharide-treated (LPS) group. LMExo: the LPS-treated plus hpMSC exosomes group. LMExoi: the LPS-treated plus inhibitor-treated hpMSC exosomes group. LEExo: the LPS-treated plus engineered exosomes group. LEExoi: the LPS-treated plus inhibitor-treated engineered exosomes group.

FIGS. 11A and 11B show the comparable efficacy of hpMSC exosomes and engineered exosomes in attenuating lipopolysaccharide-induced lung inflammation in mice. FIG. 11A shows the representative gel photography of phosphorylated nuclear factor-kB (p-NF-KB) and actin (internal standard), assayed via the method of the Simple Western and the relative band density of p-NF-KB/actin ratio in lung tissues. Data were obtained from 5 mice in each group. FIG. 11B shows the concentrations of tumor necrosis factor-α (TNF-α), interleukin-1β (IL-1β), and IL-6 in lung tissues, measured via the enzyme-linked immunosorbent assay. Data were obtained from 5mice in each group. All assays were measured at 48 hours after normal saline or lipopolysaccharide administration. Data represented as mean +standard deviations. *p<0.05, versus the Sham group. #p<0.05, versus the LPS group. †p<0.05, the LMExoi group versus the LMExo group. ∧p<0.05, the LEExoi group versus the LEExo group. Sham: the normal saline group. MExo group: the normal saline plus hpMSC exosomes group. EExo group: the normal saline plus engineered exosomes group. LPS: the lipopolysaccharide-treated (LPS) group. LMExo: the LPS-treated plus hpMSC exosomes group. LMExoi: the LPS-treated plus inhibitor-treated hpMSC exosomes group. LEExo: the LPS-treated plus engineered exosomes group. LEExoi: the LPS-treated plus inhibitor-treated engineered exosomes group.

FIGS. 12A and 12B show the comparable efficacy of hpMSC exosomes and engineered exosomes in attenuating lipopolysaccharide-induced macrophage polarization in mouse lung tissues. FIG. 12A shows the representative gel photography of hypoxia-inducible factor-1α (HIF-1α), inducible nitric oxide synthase (iNOS), and actin (internal standard), assayed via the method of the Simple Western and the relative band density of HIF-1α/actin and iNOS/actin ratios in lung tissues. Data were obtained from 5 mice in each group. FIG. 12B shows the representative microscopy images of immunohistochemistry staining assay of iNOS (marked by arrows) and the iNOS quantitative sum intensities in lung tissues. Data were obtained from 5 mice in each group. All assays were measured at 48 hours after normal saline or lipopolysaccharide administration. Data represented as mean±standard deviations. *p<0.05, versus the Sham group. #p<0.05, versus the LPS group. †p<0.05, the LMExoi group versus the LMExo group. ∧p<0.05, the LEExoi group versus the LEExo group. Sham: the normal saline group. MExo group: the normal saline plus hpMSC exosomes group. EExo group: the normal saline plus engineered exosomes group. LPS: the lipopolysaccharide-treated (LPS) group. LMExo: the LPS-treated plus hpMSC exosomes group. LMExoi: the LPS-treated plus inhibitor-treated hpMSC exosomes group. LEExo: the LPS-treated plus engineered exosomes group. LEExoi: the LPS-treated plus inhibitor-treated engineered exosomes group.

FIGS. 13A and 13B show the comparable effectiveness of hpMSC exosomes and engineered exosomes in mitigating lung oxidation induced by lipopolysaccharide in mice. FIG. 13A shows the representative gel photography of superoxide dismutase 2 (SOD2) and actin (internal standard), assayed via the method of the Simple Western and the relative band density of SOD2/actin ratio in lung tissues. Data were obtained from 5 mice in each group. FIG. 13B shows the representative microscopy images of immunohistochemistry staining assay of myeloperoxidase (MPO, marked by arrows) and the MPO quantitative sum intensities in lung tissues. Data were obtained from 5 mice in each group. All assays were measured at 48 hours after normal saline or lipopolysaccharide administration. Data represented as mean±standard deviations. *p<0.05, versus the Sham group. #p<0.05, versus the LPS group. †p<0.05, the LMExoi group versus the LMExo group. ∧p<0.05, the LEExoi group versus the LEExo group. Sham: the normal saline group. MExo group: the normal saline plus hpMSC exosomes group. EExo group: the normal saline plus engineered exosomes group. LPS: the lipopolysaccharide-treated (LPS) group. LMExo: the LPS-treated plus hpMSC exosomes group. LMExoi: the LPS-treated plus inhibitor-treated hpMSC exosomes group. LEExo: the LPS-treated plus engineered exosomes group. LEExoi: the LPS-treated plus inhibitor-treated engineered exosomes group.

FIGS. 14A and 14B show the comparable effectiveness of hpMSC exosomes and engineered exosomes in mitigating lung apoptosis induced by lipopolysaccharide in mice. FIG. 14A shows the representative gel photography of pro-apoptotic cleaved caspase 3 (19 KDa and 17 KDa) and actin (internal standard), assayed via the method of Simple Western and the relative band density of cleaved caspase 3 (19 KDa)/actin and cleaved caspase 3 (17 KDa)/actin ratios in lung tissues. Data were obtained from 5 mice in each group. FIG. 14B shows the representative microscopy fluorescent images of TUNEL assay of fragmented DNA (marked by arrows) and the TUNEL-positive cell counts in lung tissues. Data were obtained from 5 mice in each group. All assays were measured at 48 hours after normal saline or lipopolysaccharide administration. Data represented as mean±standard deviations. *p<0.05, versus the Sham group. #p<0.05, versus the LPS group. †p<0.05, the LMExoi group versus the LMExo group. ∧p<0.05, the LEExoi group versus the LEExo group. Sham: the normal saline group. MExo group: the normal saline plus hpMSC exosomes group. EExo group: the normal saline plus engineered exosomes group. LPS: the lipopolysaccharide-treated (LPS) group. LMExo: the LPS-treated plus hpMSC exosomes group. LMExoi: the LPS-treated plus inhibitor-treated hpMSC exosomes group. LEExo: the LPS-treated plus engineered exosomes group. LEExoi: the LPS-treated plus inhibitor-treated engineered exosomes group.

### DETAILED DESCRIPTION

The following examples are used for illustrating the present disclosure. A person skilled in the art can easily conceive the other effects of the present disclosure, based on the disclosure of the specification. It will be apparent that one or more embodiments may be practiced without specific details. The present disclosure can also be implemented or applied as described in different examples. It is possible to modify or alter the following examples for carrying out this disclosure without contravening its scope for different applications. Titles or subtitles may be used in this disclosure for the convenience of a reader, which shall have no influence on the scope of the present disclosure.

The practice of the present disclosure employs, unless otherwise indicated, conventional techniques of molecular biology (including recombinant techniques), microbiology, cell biology, biochemistry, immunohistochemistry, and immunology, which are well within the purview of a skilled artisan in the art. Such techniques are explained fully in the literature, such as “Molecular Cloning: A Laboratory Manual,” second edition (Sambrook, et al., 1989), Cold Spring Harbor Press; “Oligonucleotide Synthesis” (M. J. Gait, 1984); “Methods in Molecular Biology,” Humana Press; “Cell Biology: A Laboratory Notebook” (J. E. Cellis, Ed., 1998) Academic Press; “Animal Cell Culture” (R. I. Freshney, Ed., 1987); “Handbook of Experimental Immunology” (Weir, 1996); “Introduction to Cell and Tissue Culture” (J. P. Mather and P. E. Roberts, 1998); “Cell and Tissue Culture: Laboratory Procedures” (A. Doyle, J. B. Griffiths, and D. G. Newell, Eds., 1993-8); “Methods in Enzymology” (Academic Press, Inc.); “Handbook of Experimental Immunology” (D. M. Weir and C. C. Blackwell, Eds.); “Gene Transfer Vectors for Mammalian Cells” (J. M. Miller and M. P. Calos, Eds., 1987); “Current Protocols in Molecular Biology” (F. M. Ausubel, et al., Eds., 1987); “PCR: The Polymerase Chain Reaction (Mullis, et al., Eds., 1994); “Current Protocols in Immunology” (J. E. Coligan et al., Eds., 1991); “Short Protocols in Molecular Biology” (Wiley and Sons, 1999); “Immunobiology” (C. A. Janeway and P. Travers, 1997); “Antibodies” (P. Finch, 1997); “Antibodies: A Practical Approach” (D. Catty., Ed., IRL Press, 1988-1989); “Monoclonal Antibodies: A Practical Approach” (P. Shepherd and C. Dean, Eds., Oxford University Press, 2000); “Using Antibodies: A Laboratory Manual” (E. Harlow and D. Lane, Cold Spring Harbor Laboratory Press, 1999); “The Antibodies” (M. Zanetti and J. D. Capra, Eds., Harwood Academic Publishers, 1995). Particularly useful techniques for particular embodiments will be discussed in the sections that follow. Without further elaboration, it is believed that one skilled in the art can, based on the descriptions, utilize the present disclosure to its fullest extent. All publications cited herein are incorporated by reference for the purposes or subject matter referenced herein.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others. Therefore, the terms “comprise,” “comprising,” “including,” “containing,” and grammatical equivalents thereof are used in the inclusive, open sense, meaning that additional elements are not expressly mentioned but may be included. It is not intended to be construed as “consists of only.”

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The phrase “an effective amount” refers to the amount of an active ingredient that is required to result in a reduction, inhibition, or prevention of a disorder or condition, or one or more symptoms of such condition or disorder in a subject. An effective amount will vary, as recognized by those skilled in the art, depending on routes of administration, excipient usage, and the possibility of co-usage with other therapeutic treatment.

As used herein, the term “treat,” “treating,” or “treatment” refers to the application or administration of one or more active agents to a subject afflicted with a disorder, a symptom or a condition of a disease, or a progression of the disease, with the purpose to cure, heal, relieve, alleviate, alter, remedy, ameliorate, improve, or affect the disorder, the symptom or the condition of the disease, the disabilities induced by the disease, or the progression of the disease.

The term “subject” or “patient” as used herein is intended to include animals, which are suffering from or may suffer from in the near future a disease or a disorder. Examples of subjects include but are not limited to mammals, e.g., humans, apes, monkeys, dogs, cows, horses, pigs, sheep, goats, cats, mice, rabbits, rats, and transgenic non-human animals. In at least one embodiment, the subject is a human, e.g., a human suffering from, at risk of suffering from, or potentially capable of suffering from a disease or a disorder.

The term “in vitro” and “ex vivo” as employed herein refers to laboratory work not performed in a human or animal body.

The term “in vivo” as employed herein refer to work/testing/treatment in a living organism, e.g., a human or an animal, such as a human.

Extracellular vesicles are a broad term to describe all secreted membrane vesicles. As employed herein, the term “extracellular vesicles” include exosomes, microvesicles (also referred to as microparticles), ectosomes, matrix vesicles, calcifying vesicles, prostasomes, oncosomes, retrovirus-like particles, bacterial extracellular vesicles, intraluminal vesicles, and apoptotic bodies. Extracellular vesicles generally have a diameter in the range of from 10 nm to 5,000 nm. As used herein, the term “exosome” refers to a cell-derived small vesicle (between 20 nm to 300 nm in diameter, e.g., 40 nm to 200 nm in diameter) comprising a membrane that encloses an internal space, and which is generated from said cell by direct plasma membrane budding or by fusion of the late endosome with the plasma membrane. The exosome comprises lipids or fatty acids and polypeptides and further comprises the miRNA described herein as a payload. The exosome can be derived from a producer cell and isolated from the producer cell based on its size, density, biochemical parameters, or a combination thereof. Exosomes can be directly loaded with exogenous nucleic acids by electroporation, lipofection, sonication, or contact with calcium chloride. Alternatively, purified exosomes may be loaded ex vivo by, for example, electroporation.

As used herein interchangeably, the term “microRNA” or “miRNA” refers to the unprocessed or processed RNA transcript from a miRNA gene. MicroRNAs (miRNAs) are non-coding RNAs (typically 19 to 25 nucleotides in length) that regulate gene expression by inducing translational inhibition or cleavage of their target mRNA through base pairing to partially or fully complementary sites.

The exosomes of the present disclosure can be produced from a cell grown in vitro or a body fluid of a subject. When exosomes are produced from in vitro cell culture, various producer cells, e.g., immune cells such as macrophage cell lines (e.g., RAW264.7 cells and THP-1 cells), HEK293 cells, Chinese hamster ovary (CHO) cells, or mesenchymal stem cells (MSCs), can be used.

The pharmaceutical compositions described herein may be administered in dosages sufficient to treat a disease in a subject in need thereof. The specific dosages of the pharmaceutical compositions described herein and administered to a given subject will depend on factors such as the route of administration and physical characteristics of the subject (including health status) and so forth. For example, the appropriate dosage of a given pharmaceutical composition comprising the miRNA described herein may depend on a variety of factors including, but not limited to, a subject's physical characteristics (e.g., age, weight, and gender), the progression (i.e., pathological state) of a disease, and other factors that will be readily recognized by one skilled in the art. Various general considerations that may be considered when determining an appropriate dosage are described, for example, in Gennaro et al. (Eds), (1990), “Remington's Pharmaceutical Sciences,” Mack Publishing Co., Easton, Pennsylvania, USA and Gilman et al. (Eds), (1990), “Goodman and Gilman's: The Pharmacological Bases of Therapeutics,” Pergamon Press. Those of ordinary skill in the art will be able, by routine experimentation, to determine an effective, non-toxic amount of the pharmaceutical compositions described herein to include in a dosage or in a series of dosages to achieve the desired therapeutic effect.

In therapeutic applications, the treatment would be for the duration of the disease state or condition. Further, it will be apparent to one of ordinary skill in the art that the optimal quantity and interval of individual dosages will be determined by the nature and extent of the disease state or condition being treated, the form, route and site of administration, and the nature of the particular individual being treated. Such optimal conditions can also be determined using conventional techniques.

In many instances, it will be desirable to have several or multiple administrations of a pharmaceutical composition described herein. For example, they may be administered 1, 2, 3, 4, 5, 6, 7, 8, 9, 10, or more times. The administrations may be from about one to about twelve-week intervals, e.g., from about one to about four-week intervals. It will also be apparent to one of ordinary skill in the art that the optimal course of treatment can be ascertained using conventional course of treatment determination tests.

The pharmaceutical compositions of the present disclosure may be administered in any suitable way, for example, intravenously, buccally, parenterally, intranasally, orally, sublingually, or topically. Accordingly, the administration may be topical, pulmonary (e.g., by inhalation or insufflation of aerosols or powders with a nebulizer), intranasal, intratracheal, epidermal, transdermal, oral, or parenteral administration. Parenteral administration includes intravenous, intraarterial, subcutaneous, intraperitoneal, or intramuscular injection or infusion, or intracranial (e.g., intraparenchymal, intrathecal, or intraventricular) administration. In at least one embodiment, the pharmaceutical composition is adapted for intranasal administration. In at least one embodiment, the pharmaceutical composition of the present disclosure is formulated as a direct-acting nasal spray. In at least one embodiment, the nasal spray of the present disclosure can be self-administered at point-of-care.

### EXAMPLES

Exemplary embodiments of the present disclosure are further described in the following examples, which should not be construed to limit the scope of the present disclosure.

### Preparation Example 1. Preparation of Engineered Exosomes Loaded with hsa-let-7i-5p miRNAs by Electroporation

Murine macrophage cell line RAW264.7 was used for exosome preparation. Murine macrophage cell line RAW264.7 (murine macrophage-like cells; ATCC, USA) was cultured in Dulbecco's modified Eagle's medium (DMEM; Life Technologies) supplemented with 10% fetal bovine serum (FBS) and 1% antibiotics (penicillin/streptomycin; Life Technologies). Cell culture dishes were maintained in a 37° C. incubator with 5% CO₂, with the culture media changed every four days, and regular passages were performed. Cell cultures were controlled within 8 to 10 passages for exosome production. Transfected RAW264.7 cell lines expressing hsa-let-7i-5p were established following the methods described by Madhyastha et al. (Madhyastha R. et al., Inflammation. 2021 August; 44(4): 1274-1287). Briefly, hsa-let-7i-5p plasmid DNA (SC400011) was purchased from OriGene Technologies, Inc. One microgram of the hsa-let-7i-5p plasmid DNA was diluted in OptiMEM (Invitrogen, NY, USA) and electroporated into RAW264.7 cells using 250 V, 5 pulses, each with a length of 100 ms. The cell suspension after electroporation was transferred to a 6 cm culture dish containing 2 mL DMEM culture medium and cultured at 37° C. in a CO₂ incubator for 24 hours. Subsequently, cells were observed under a fluorescence microscope at 200× magnification, and further quantitative culturing was conducted by replacing the medium with G418 antibiotic-containing DMEM medium. Following literature procedures (McDonald et al., Pain. 2014 August; 155(8): 1527-1539), exosomes were isolated from the murine macrophage cell line RAW264.7. After 48 hours of incubation in RAW264.7 culture medium, the medium was collected and subjected to low-temperature centrifugation. The supernatant was collected, filtered through a 0.22 μm filter (Merck Millipore), and then subjected to ultracentrifugation at 100,000 g for 90 minutes at 4° C. to pellet the exosomes. After carefully removing the supernatant, exosome-containing pellets were resuspended in 100 μL PBS (phosphate-buffered saline) and stored at −80° C.

To characterize the engineered exosome particles obtained, the morphology of exosomes was evaluated by transmission electron microscope (Hitachi HT-7700; Hitachi, Ltd., Tokyo, Japan), and sizing was calculated by NanoSight NS300 particle size analyzer (NTA, Malvern Panalytical, Malvern, UK) following the manufacturer's protocols. Then, exosome markers including CD63 and CD9 were detected by conventional immunoblotting assay. For example, equal amounts of proteins (100 μg) were separated by electrophoresis and then transferred to nitrocellulose membranes (Bio-Rad Laboratories, Hercules, CA, USA). The membranes were then incubated with primary antibodies including anti-CD63 antibody (Proteintech, USA, 25682-1-AP) and anti-CD9 antibody (Proteintech, USA, 20597-1-AP). Bound antibody was detected through chemiluminescence (ECL Plus kit; Amersham, Buckinghamshire, UK). Protein band density was measured using densitometry (ImageJ).

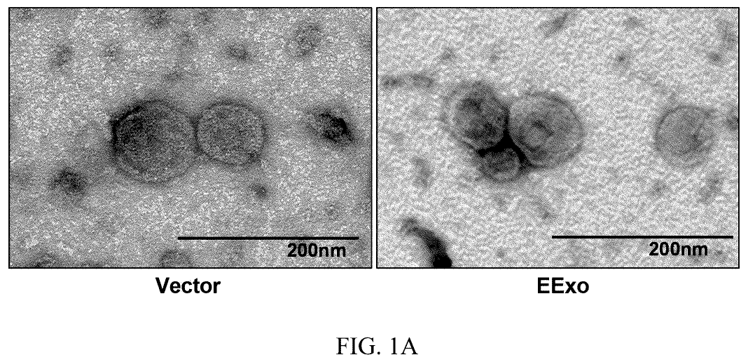

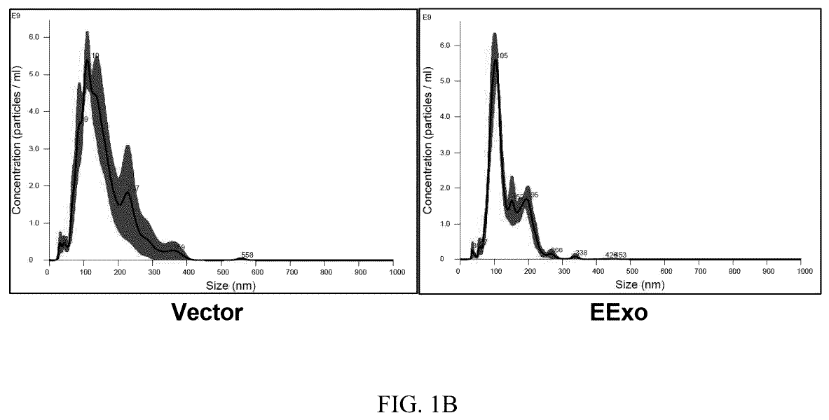

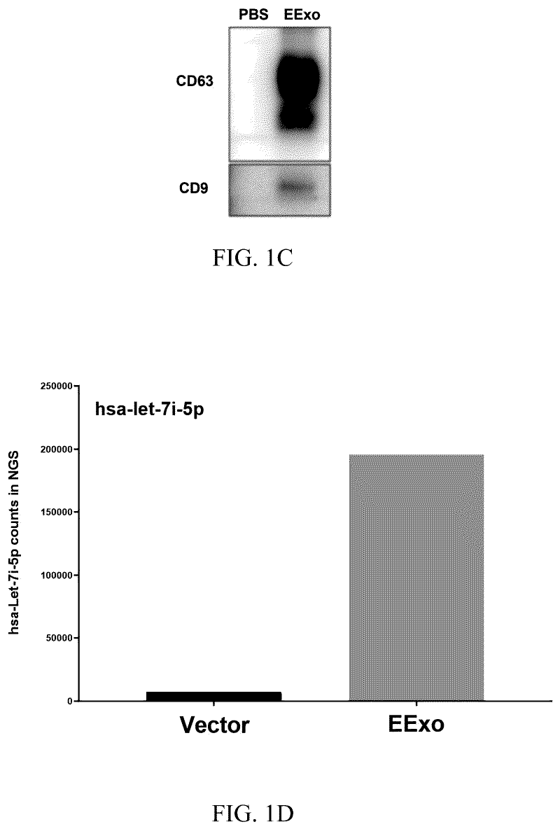

For the engineered exosomes prepared in this example, transmission electron microscopy verified oval-shaped double-layer structures (FIG. 1A), and nanoparticle tracking assays confirmed a particle size of approximately 100 nanometers (FIG. 1B). Also, immunoblotting assays validated the presence of surface markers CD63 and CD9 (FIG. 1C).

Additionally, the abundance of hsa-let-7i-5p miRNA in the engineered exosomes was analyzed through the next-generation sequencing assay. Briefly, 100 ng of total RNAs were used as input material for the small RNA sample preparations. Sequencing libraries were generated using QIAseq miRNA Library Kit (QIAGEN, Germany), following the manufacturer's recommendations. Briefly, 3′- and 5′-adaptors were directly and specifically ligated to 3′- and 5′-end of small RNAs, respectively. Then, first-stranded cDNA was synthesized using QIAseq miRNA NGS RT Enzyme and RT primer. After PCR amplification, the library was size-selected with 170 to 200 bp by QIAseq beads. The quality and quantity of purified libraries were assessed on the Qsep400 system (Bioptic Inc., Taiwan) and Qubit 2.0 Fluorometer (Thermo Scientific, Waltham, MA, USA). The qualified libraries were then sequenced on Illumina NovaSeq 6000 platform with trimmed 75 bp single-end reads generated by Genomics, BioSci & Tech Co., New Taipei City, Taiwan. The adapter sequences in raw sequenced data were removed using Trim Galore! (v0.6.6), and mature and hairpin miRNAs (miRBase v.22.1) were mapped to reference genome with Bowtie (v1.3.0) to obtain proper miRNA reads. After the alignment step, bam files were processed using Samtools (v1.12), and the expression profile of miRNAs was calculated and normalized by using edgeR (v3.26.5). All the differentially expressed miRNAs (DEmiRNAs) were identified using DEGSeq (v1.48.0). As shown in FIG. 1D, the abundance of hsa-let-7i-5p miRNA was confirmed in the engineered exosomes.

### Preparation Example 2. Preparation of Engineered Exosomes Loaded with hsa-let-7i-5p miRNAs by Transfection

RAW264.7 cells and human placenta mesenchymal stem cells (hpMSC) were used to prepare exosomes for comparison. RAW264.7 cells were purchased from the Bioresource Collection and Research Center, Taiwan (Hsinchu, Taiwan). Human placenta-derived MSC was provided by Professor Yen-Huan Huang from Taipei Medical University, Taipei, Taiwan (N202101014: approval from the Joint Institutional Review Board of Taipei Medical University regarding the human study of human placenta acquisition and hpMSC isolation). RAW264.7 cells and hpMSC were maintained as previously reported (Chiang, M. D. et al., Antioxidants. 2022, 11, 615; Chang, C. Y. et al., Pharmaceuticals. 2021, 15, 36; Lin, C. Y. et al., Br. J. Anaesth. 2010, 104, 44-51).

RAW264.7 cells were then modified genetically to overexpress hsa-let-7i-5p. Briefly, confluent RAW264.7 cells were cultured in Dulbecco's modified Eagle's medium 126 (DMEM; Life Technologies), supplemented with 10% fetal bovine serum and 1% penicillin-streptomycin (all from Life Technologies, USA), and maintained in a humidified incubator with 5% CO₂ in air. After adherence, the RAW264.7 cells were cultured in serum-free medium (Life Technologies) for 30 minutes, followed by transfection with plasmids (2 μg) overexpressing hsa-let-7i-5p (pCMV-let-7i-5p, SC400011; OriGene Technologies, USA). After reaction for 5 minutes, serum-free medium (205 μL) containing Lipofectamine 3000 (5 μL) (both from Thermo Fisher, USA) was added to the mixtures. The hsa-let-7i-5p-overexpressing stable cell lines were then screened with neomycin.

To isolate engineered exosomes from RAW264.7 cells overexpressing hsa-let-7i-5p (engineered exosomes) and exosomes from hpMSC (hpMSC exosomes), culture medium was harvested and centrifuged (Beckman Coulter Allegra X-15R centrifuge, 300 g, 4° C., 10 minutes). Supernatant was filtered (0.22 mm filters, Millipore, USA) and then ultracentrifuged (Beckman Coulter Optima L-80XP Ultracentrifuge, 100,000 g, 4° C., 90 minutes, with a Type 50.2 Ti rotor, k-factor: 157.7) to pellet exosomes. The pellets were resuspended and then pooled, ultracentrifuged, resuspended, purified, and again ultracentrifuged. The top fractions of the gradient were then collected, diluted, and centrifuged. The crude exosome-containing pellets were resuspended in 1 mL of ice-cold PBS and pooled. A second round of ultracentrifugation was performed, and the resulting exosome pellets were resuspended again in 500 μL of phosphate buffered saline (PBS, Life Technologies) and stored (−80° C.).

The obtained exosomes were then characterized and analyzed by observing their morphology, analyzing their size and markers, in addition to the amount of hsa-let-7i-5p in the exosomes. Morphology of the isolated engineered exosomes and hpMSC exosomes was confirmed using transmission electron microscopy (TEM). For TEM, exosome suspensions (3 μL) were fixed (50 μL, 2% paraformaldehyde, Sigma-Aldrich) and then transferred onto 2 Formvar-carbon coated electron microscopy grids, followed by observation with a transmission electron microscope (JEM 1400 series, JEOL, USA). For exosome particle sizing analysis, the NanoSight NS300 instrument (Nanosight, Malvern Panalytical, UK) was used according to the manufacturer's protocols. For the analysis of exosome markers, ALIX and CD9, a capillary-based Western blot analysis (the Simple Western method) was conducted using the WES system (ProteinSimple, Santa Clara, CA, USA) (Rumbaugh, G, et al., Methods Mol. Biol. 2011, 670, 263-74). Protein samples were diluted and prepared in accordance with the manufacturer's protocol. After denaturation (95° C., 5 min), the samples were loaded onto the plate. Electrophoretic separation, antibody incubation, and chemiluminescence detection were performed within the WES system using default settings.

Primary antibodies targeting ALIX (anti-ALIX antibody, ab235377; Abcam, Cambridge, UK) and CD9 (anti-CD9 antibody, IR300-981; iReal Technology, Hsinchu, Taiwan) were utilized. The digital image was analyzed using Compass software (ProteinSimple), with quantified data of the detected proteins reported in terms of molecular weight and signal/peak intensity.

To analyze the miRNA hsa-let-7i-5p levels in exosomes, droplet digital PCR (ddPCR) was utilized. Total RNA was extracted from hpMSC exosomes and engineered exosomes using the miRNeasy Serum/Plasma Kit (Qiagen, Hilden, Germany), following the manufacturer's recommended procedure. Reverse transcription was conducted with the TaqMan MicroRNA Assay (Thermo Fisher, Waltham, MA, USA) as per the manufacturer's instructions. The miRNA copy numbers were then analyzed using the QX200 ddPCR system (Bio-Rad, Hercules, CA, USA). In accordance with the manufacturer's protocol, the reverse transcription products, primers, master mix, and mineral oil were loaded into a droplet generator to generate thousands of droplets. PCR amplification was carried out with the TaqMan MicroRNA Assay (Thermo Fisher, Waltham, MA, USA). Subsequently, the droplets were aspirated and read using the Droplet Reader (Bio-Rad, Hercules, CA, USA). Data analysis was performed using QUANTASOFT analysis software (Bio-Rad, Hercules, CA, USA). Technical support for this study was provided by the National Genomics Center for Clinical and Biotechnological Applications, National Yang Ming Chiao Tung University, Taipei, Taiwan.

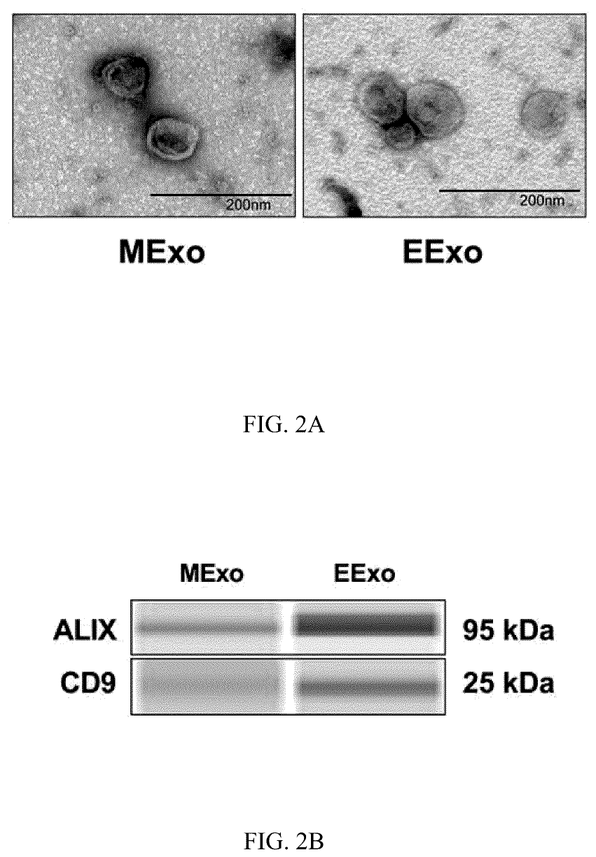

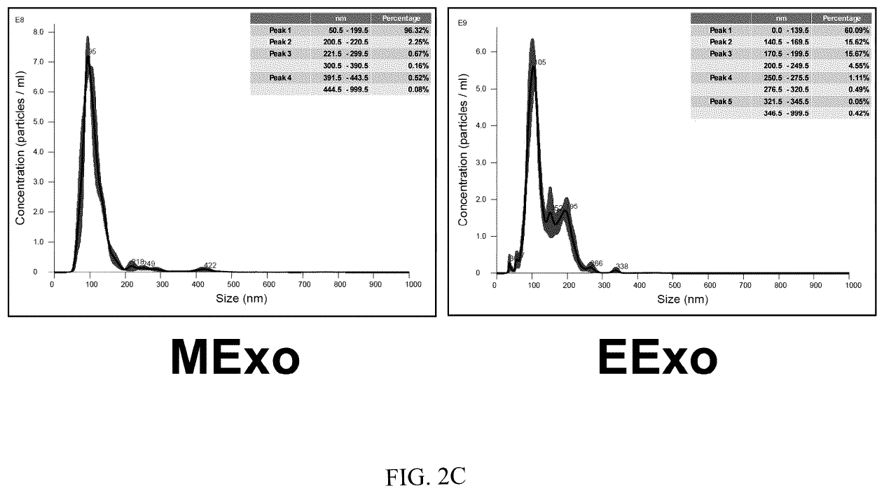

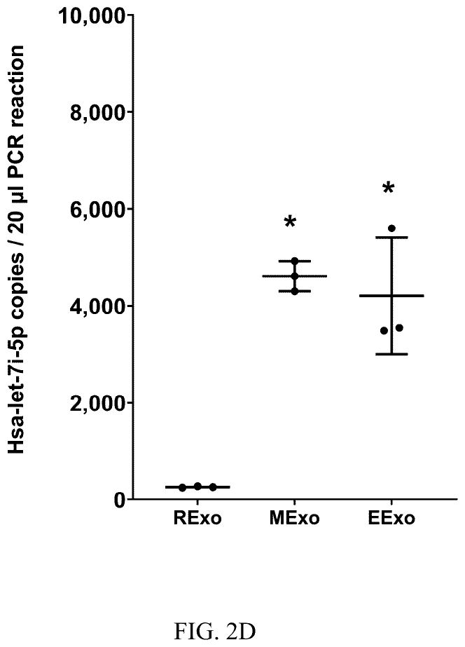

FIGS. 2A to 2D show the results of the above analysis. Both hpMSC exosomes and engineered exosomes exhibited a characteristic double layered cup-shaped morphology under TEM, as depicted in FIG. 2A. Immunoblotting assays confirmed the presence of positive markers ALIX and CD9 (FIG. 2B). Nanoparticle tracking analysis revealed particle sizes of approximately 50 to 200 nm for both types of exosomes (FIG. 2C). Furthermore, as shown in FIG. 2D, miRNA concentrations of hsa-let-7i-5p in both hpMSC exosomes and engineered exosomes were as many as 4,610±310 and 4,207±1207 copies, respectively, and both were significantly higher than that in exosomes isolated from RAW264.7 cells without genetic modification (259±14 copies, both p<0.001).

### Example 1. Engineered Exosomes Have Suitable Biodistribution and Pharmacokinetics Properties for Treating Lung Diseases

A set of mice were used for monitoring biodistribution of exosomes. To facilitate imaging, exosomes were incubated with 5 μM of Cy7 mono-N-hydroxysuccinimide (NHS) ester (Amersham Biosciences, Little Chalfont Bucks, UK) for 30 min at 37° C. to prepare Cy7-labeled exosomes (1×10⁸).

Mice were administered with the Cy7-labeled exosomes both intraperitoneally and intratracheally. At 2, 24, and 48 hours after Cy7-labeled exosomes injection, the mice were sacrificed after blood drawing via the cardiac puncture, and the organs including heart, lungs, liver, kidneys, spleen, and bladder were harvested. Distribution of the engineered exosomes in different organs was measured and recorded. Bioluminescence image assay was performed using an in vivo imaging system (IVIS Lumina XRMS; PerkinElmer, Waltham, MA, USA), and the images were then analyzed using the Living Image software (PerkinElmer).

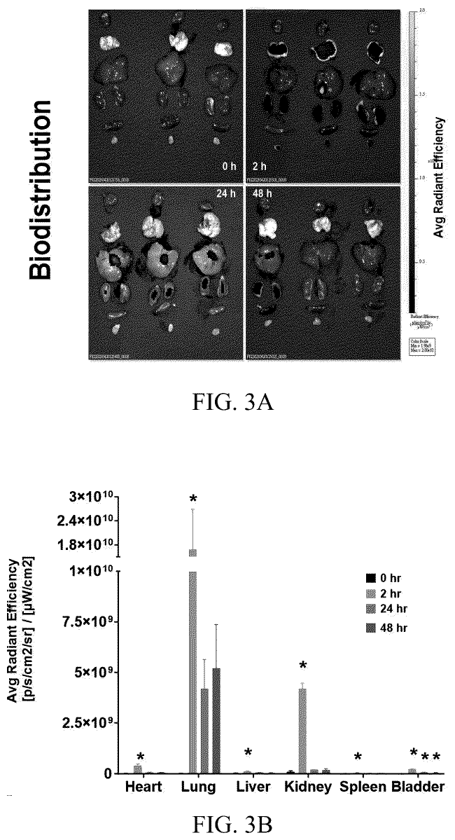

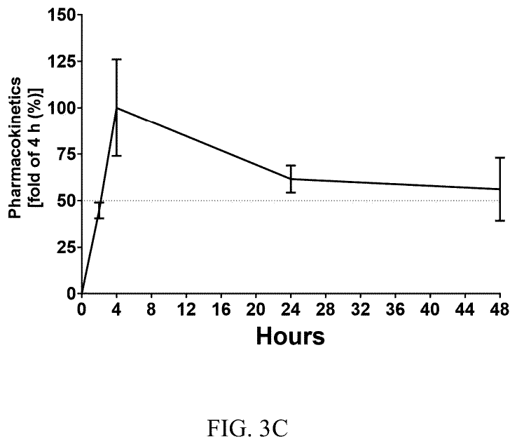

In vivo imaging demonstrated significant biodistribution of engineered exosomes in lung tissues, and the pharmacokinetic assay revealed an approximate in vivo half-life of 48 hours for intraperitoneal administration of the engineered exosomes (1×10⁹ particles/mouse) (FIGS. 3A to 3C).

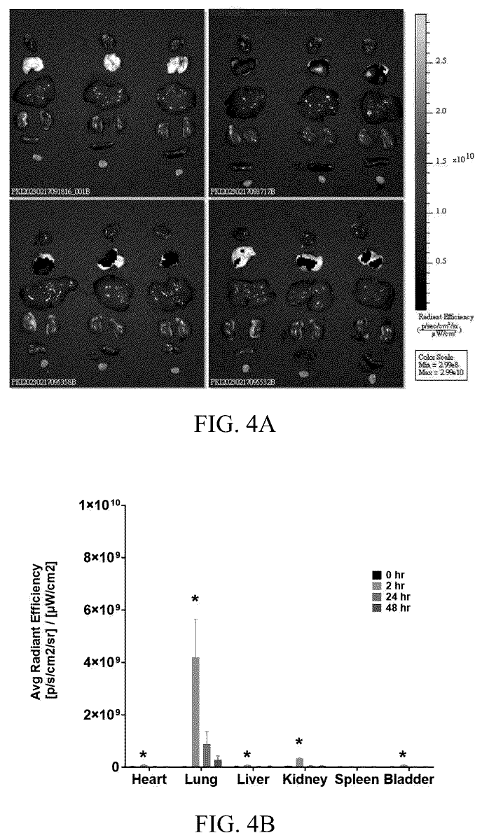

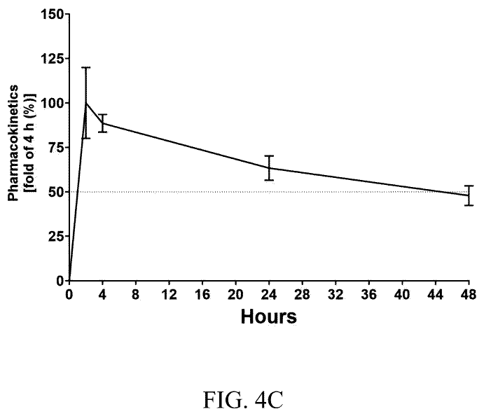

Similar results were observed with intratracheal administration of the engineered exosomes, as the in vivo imaging also demonstrated significant biodistribution of engineered exosomes in lung tissues, and the pharmacokinetic assay also revealed an approximate in vivo half-life of 48 hours for intratracheal administration of the engineered exosomes (1×10⁹ particles/mouse) (FIGS. 4A to 4C).

### Example 2. Engineered Exosomes and hpMSC Exosomes have Comparable Biodistribution and Pharmacokinetics Properties

An independent cohort of 24 mice was used for this assay. In brief, the mice were divided into two groups (n=12 in each group). The first group of mice received intraperitoneal injections of hpMSC exosomes labeled with Cy7 mono NHS ester (Amersham Biosciences, UK) at a dose of 1×10⁸ particles per mouse. The second group of mice received engineered exosomes, also labeled with Cy7 mono NHS ester (Amersham Biosciences, UK), at a dose of 1×10⁹ particles per mouse. Three mice from each group were sacrificed at 0, 2, 24, and 48 hours via decapitation, and all vital organs were collected. The Cy7 signal was detected using a bioluminescence imaging assay (IVIS Lumina XRMS and Living Image software; PerkinElmer, USA).

An assay of pharmacokinetics was performed with another independent cohort of 6 mice, which were also divided into two groups (n=3 in each group). In brief, the first group of mice received intraperitoneal injections of hpMSC exosomes labeled with Cy7 mono NHS ester (Amersham) (1×10⁸ particles per mouse), and the second group of mice received engineered exosomes, also labeled with Cy7 mono NHS ester (Amersham) (1×10⁹ particles per mouse). Serial blood sampling was performed in the 3 mice from each group, through submandibular vein puncture before administration (0 h) and at 2, 4, 24, and 48 hours after administration. The fluorescence of each sample was measured using the SpectraMax M5 microplate reader (Molecular Devices, USA), with an excitation at 756 nm and an emission peak at 779 nm, to facilitate pharmacokinetic analysis of hpMSC exosomes and engineered exosomes, respectively.

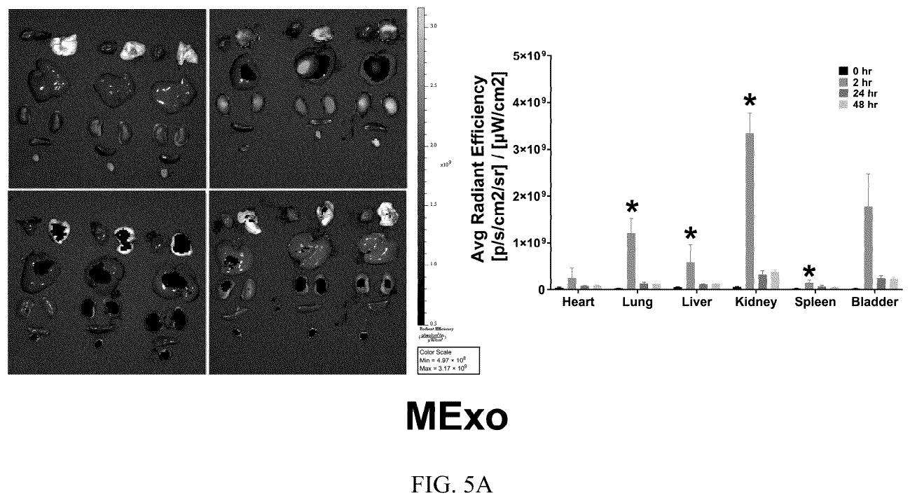

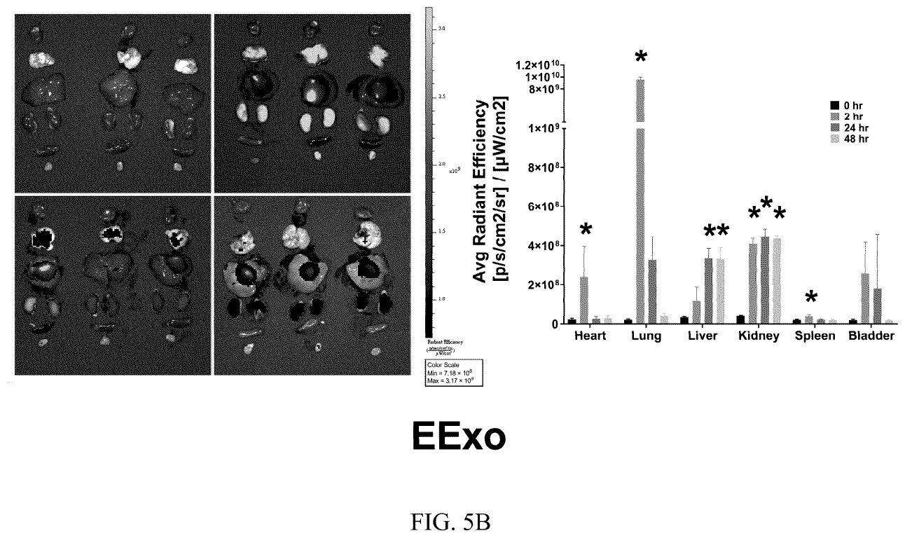

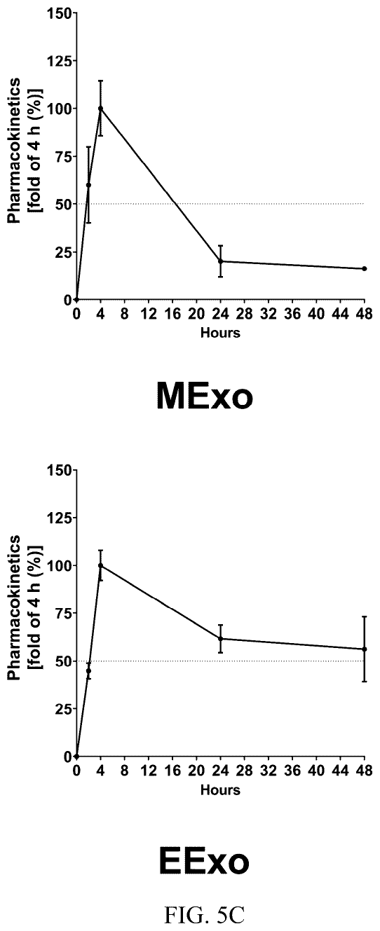

The biodistribution and pharmacokinetic analysis results were shown in FIGS. 5A to 5C. In mice receiving Cy7-conjugated hpMSC exosomes, bioluminescence imaging revealed significantly higher Cy7 signal intensities in the lungs, liver, kidneys, and spleen at 2 hours post-administration compared to baseline (all p<0.05) (FIG. 5A). However, at 24 and 48 hours post-administration, Cy7 signal intensities in these organs were not significantly different from baseline. It was found that there were no significant differences in Cy7 signal intensities in the heart and bladder at any time point post-administration. These findings demonstrate significant biodistribution of hpMSC exosomes in the lungs, liver, kidneys, and spleen shortly after administration, with a duration of less than 24 hours. In mice receiving Cy7-conjugated engineered exosomes, bioluminescence imaging showed significantly higher Cy7 signal intensities in the heart, lungs, kidneys, and spleen at 2 hours post-administration compared to baseline (all p<0.05) (FIG. 5B). However, at 24 and 48 hours, Cy7 signal intensities in the heart, lungs, and spleen were not significantly different from the baseline, while those in the liver and kidneys remained significantly elevated (all p<0.05). Similar to hpMSC exosomes, there were no significant differences in Cy7 signal intensities in the bladder at any time point post-administration. These data indicate significant biodistribution of engineered exosomes in the heart, lungs, liver, kidneys, and spleen after administration.

Pharmacokinetic analyses revealed that plasma concentrations of both hpMSC and engineered exosomes peaked at 4 hours post-administration, with an approximate half-life of 16 hours for hpMSC exosomes and 48 hours for engineered exosomes (FIG. 5C).

### Example 3. Engineered Exosomes Alleviate Sepsis-Induced Lung Injury

For the murine sepsis study, intraperitoneal injection of 25 mg/kg lipopolysaccharide (LPS, E. coli 0127: B8 endotoxin; Sigma-Aldrich, St. Louis, MO, USA) was employed to induce sepsis in mice. In brief, adult male C57BL/6 mice were randomly assigned to two groups: one receiving intraperitoneal administration of LPS (LPS group); and the other receiving intraperitoneal administration of LPS along with intraperitoneal administration of engineered exosomes loaded with hsa-let-7i-5p miRNA (1×10⁹ particles/mouse; the LPSEExo group). Engineered exosomes were administered at 2 h and 26 h after LPS administration. Controls were concurrently run that include a mice group administered with normal saline (Sham) and a mice group receiving only intraperitoneal administration of engineered exosomes loaded with hsa-let-7i-5p miRNA (1×10⁹ particles/mouse; the EExo group).

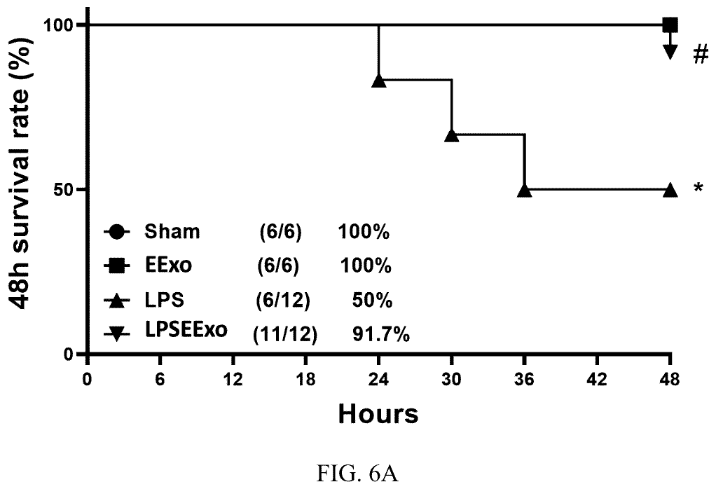

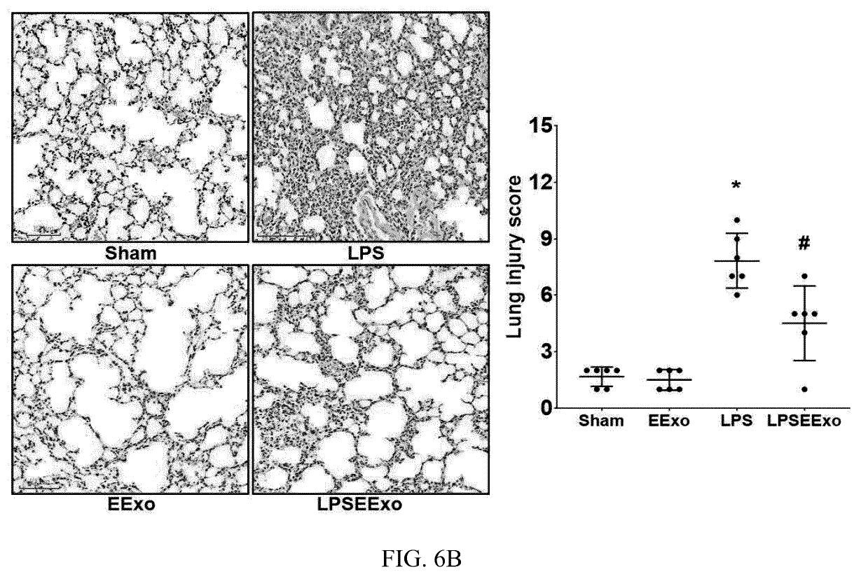

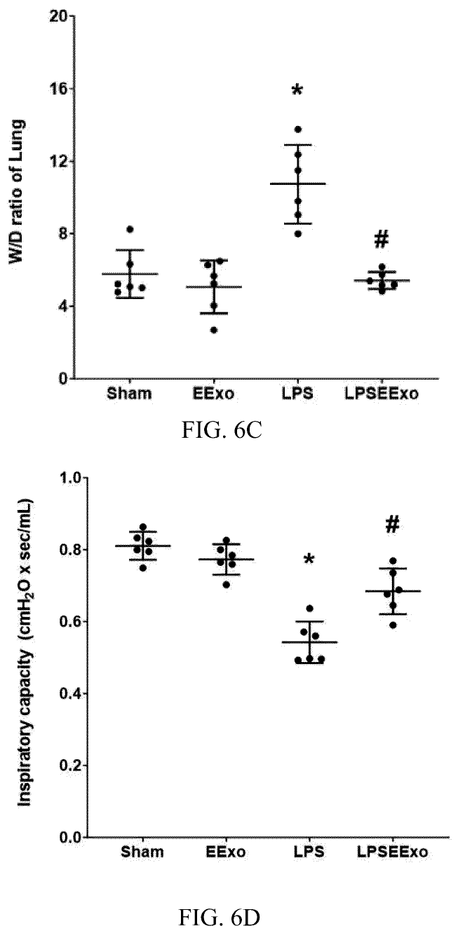

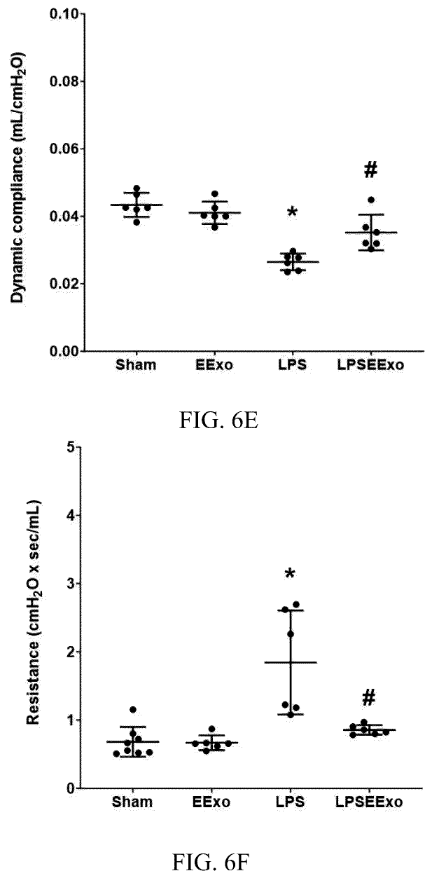

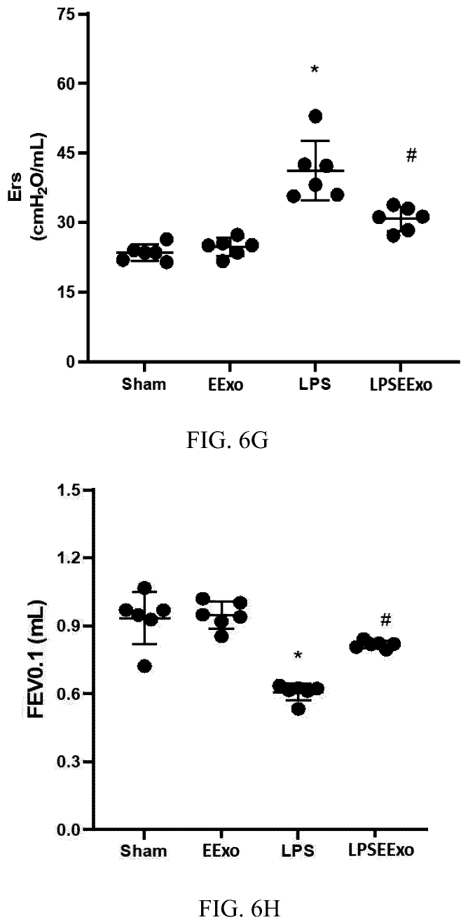

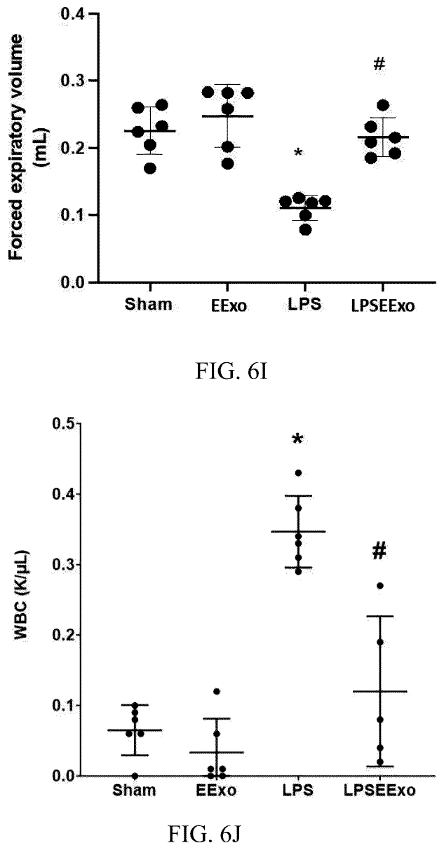

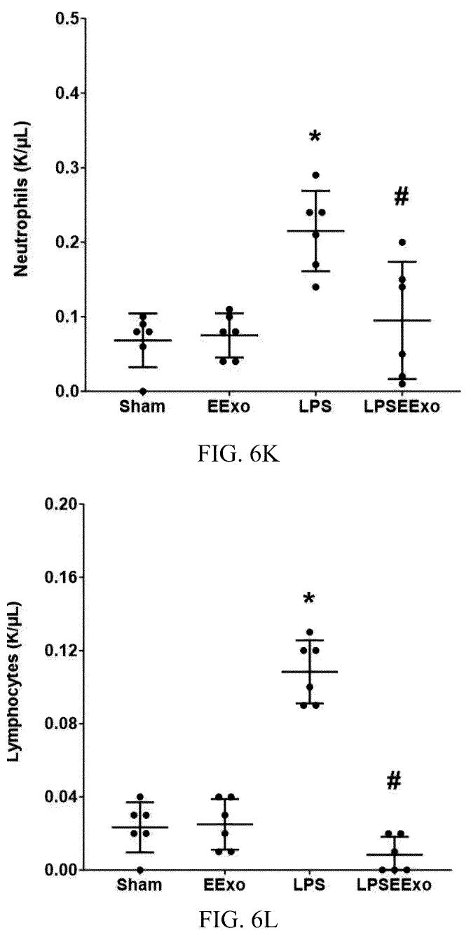

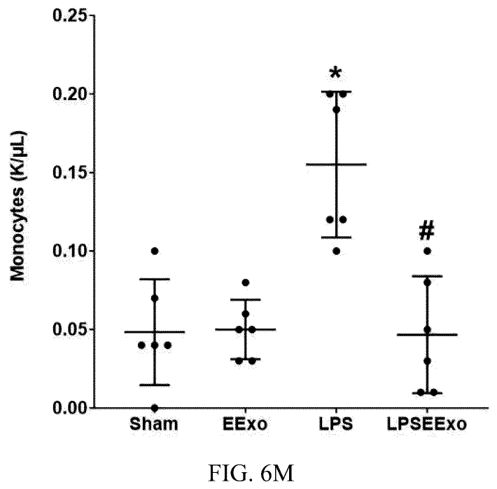

Following close observation for 48 hours, the 48-hour survival rate was determined. The 48-hour survival rate in the LPSEExo group was significantly higher than that in the LPS group (p=0.0204), as shown in FIG. 6A.

The surviving mice were subsequently euthanized to assess and compare the degree of lung injury in each group.

First, a set of mice in each group received 10% formalin solution (Sigma-Aldrich) perfusion via the tracheostomy tube, and the lung tissues were then removed and harvested for histologic analysis. The formalin-infused lung tissues were placed in paraffin wax, followed by sequential sectioning and then stained with hematoxylin and eosin (HE). Lung injury was evaluated according to the histological characteristics, including alveolar wall edema, hemorrhage, vascular congestion, and polymorphonuclear leukocyte (PMN) infiltration, using a light microscope. Each of the histological characteristics was further rated, based on a scale of 0 to 5 (normal to severe). The sum (i.e., the lung injury score) was then calculated to determine lung injury levels. The histological and lung injury score assays based on histological characteristics evaluation with hematoxylin and eosin (HE) stains showed that the lung injury level in the LPS group was significantly higher than that in the LPSEExo group (p=0.0012; FIG. 6B).

Further, mice from each group also received a midline laparotomy and sternotomy to expose the abdominal aorta and the lungs. After euthanasia, the trachea was ligated, and the left and right lungs were freshly dissected and removed. The freshly harvested lung tissues were divided and collected. Half of the collected lung tissue samples were snap frozen in liquid nitrogen and stored at −80° C. for later analysis. The other half of the collected lung tissue samples were used for Wet/Dry (W/D) ratio assay, which was conducted to determine lung water content, a lung injury marker. In brief, the freshly harvested lung tissue samples were weighed, placed in the oven (80° C.) for 24 h, and then weighed again. The W/D ratios of the lung tissue samples were then calculated. The result shows that the wet/dry weight ratio was significantly higher in the LPS group than in the LPSEExo group (p<0.0001; FIG. 6C).

For assays to assess lung function, the anesthetized mice received a tracheostomy, and a 20G catheter (B. Braun, Melsungen, Germany) was inserted as the tracheostomy tube. The tube was then connected to a computerized small animal ventilator (flexi Vent FX; SCIREQ Inc., Montreal, Canada). The mechanical ventilation was set at a ventilation rate of 150 breaths/min and a tidal volume of 0.2 mL. The airway resistance and dynamic compliance were recorded using a flexiWare 8 System (SCIREQ Inc., Montreal, Canada). Inspiratory capacity (IC), resistance (Rrs), dynamic compliance (Crs), elastance (Ers), forced expiratory volume in 0.1 sec (FEV0.1), and forced expiratory volume (FEV) were obtained. As shown in FIGS. 6D to 6I, lung function assays revealed significantly lower inspiratory capacity, dynamic compliance and forced expiratory volume and significantly higher resistance and elastance in the LPS group as compared to the controls (the Sham and EExo groups), while administration of engineered exosomes loaded with hsa-let-7i-5p miRNA to the mice of LPSEExo group reversed the lung injuries and observed increase in inspiratory capacity, dynamic compliance and forced expiratory volume, and decrease in resistance and elastance.

To estimate inflammation state of lung tissues, the bronchoalveolar lavage fluids (BALFs) were collected and analyzed. Briefly, a set of anesthetized mice from each group received 5 times lavage with 1 mL sterile normal saline via the tracheostomy tube. The BALF samples were analyzed with IDEXX ProCyte Dx automated hematology instrument (software version 00-33_51). ProCyte reports a 5-part feline automated differential count of total neutrophils, lymphocytes, monocytes, eosinophils, and basophils. As a result, the total and differential cell counts in the BALFs of the LPS group were also significantly higher than those of the control groups (Sham and EExo groups), while administration of engineered exosomes loaded with hsa-let-7i-5p miRNA to the mice of LPSEExo group reversed the inflammation and lowered the cell counts (p<0.0001, p=0.0044, p<0.0001, and p=0.0002, respectively; FIGS. 6J to 6M).

### Example 4. Engineered Exosomes Alleviate Aspiration Pneumonia-Induced Lung Injury

For the murine aspiration pneumonia study, intratracheal administration of gastric content to induce aspiration pneumonia in mice was employed. To simulate gastric content, a mixture comprising a xanthan gum-based thickener (12 mg/mL), pepsin (2 mg/mL), and lipopolysaccharide (2.5 mg/mL) was prepared, and the pH was adjusted to 1.6. Adult male C57BL/6 mice were randomly assigned to two groups: one receiving intratracheal administration of the gastric content mimic (AP group); and the other receiving intratracheal administration of the gastric content mimic along with engineered exosomes loaded with hsa-let-7i-5p miRNA (1×10⁹ particles/mouse; the APEExo group). Engineered exosomes were administered intratracheally at 2 h and 26 h after the aspiration of gastric content mimic mixtures. Control groups were also concurrently run that include a mice group administered with normal saline (Sham) and a mice group receiving only intratracheal administration of engineered exosomes loaded with hsa-let-7i-5p miRNA (1×10⁹ particles/mouse; the EExo group).

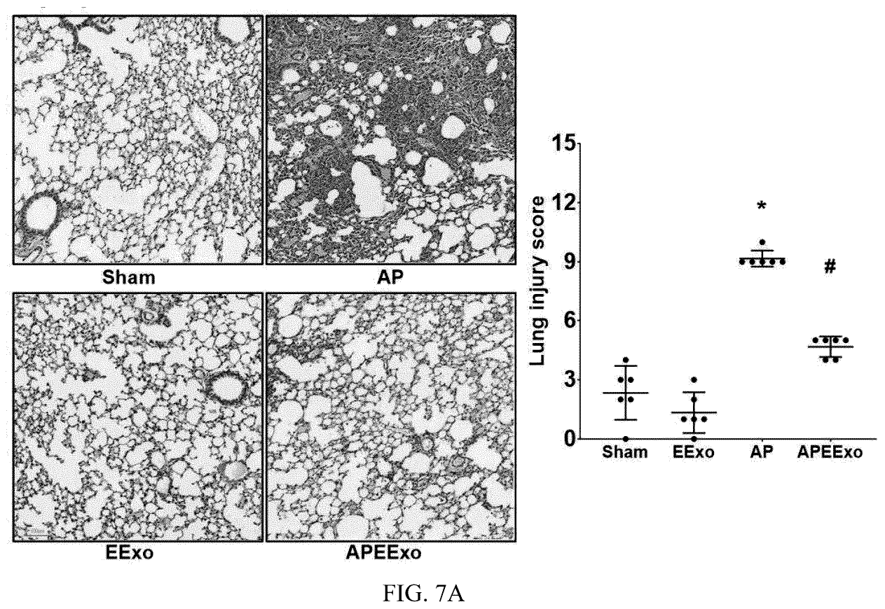

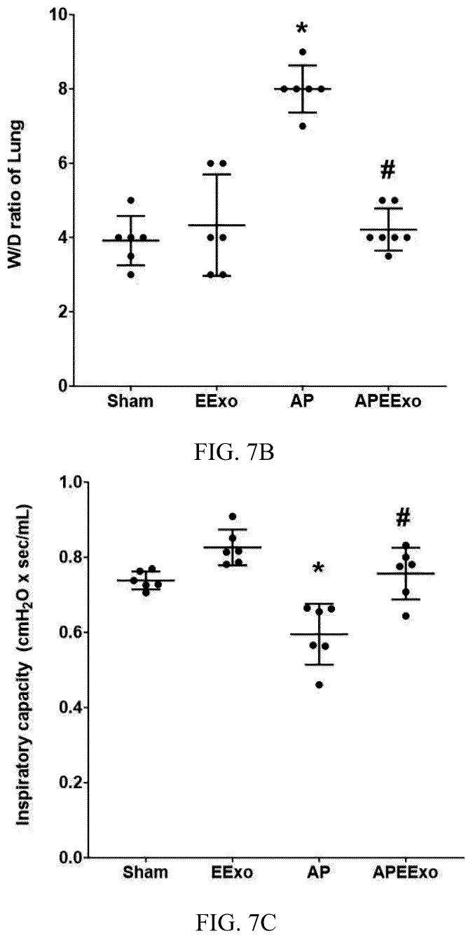

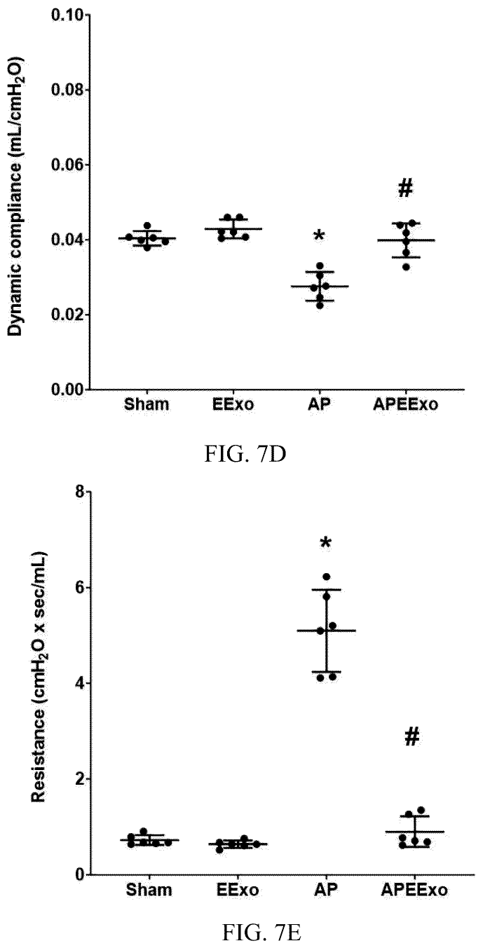

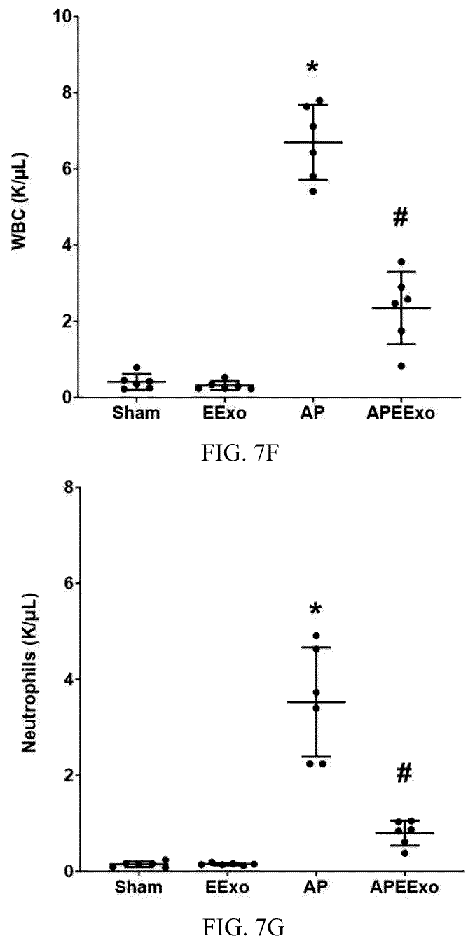

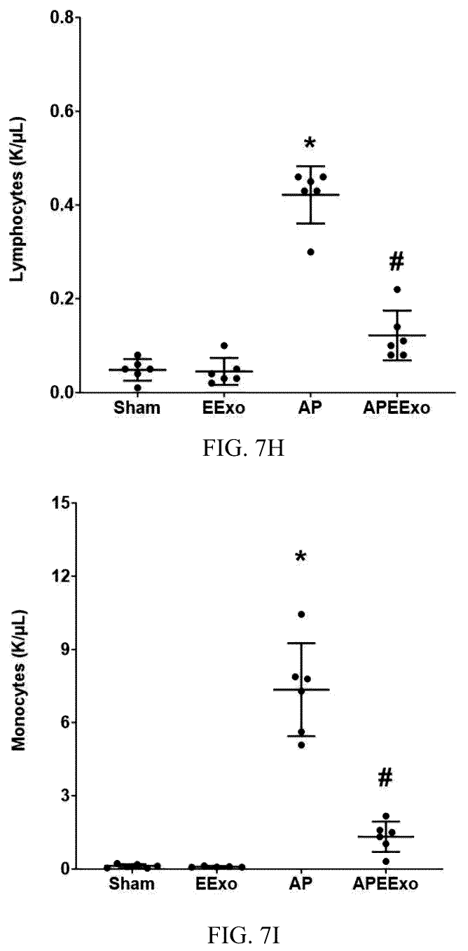

Following close observation for 48 hours, all mice were euthanized, and the degree of lung injury in each group was assessed and compared, following the same method described above. The histological and lung injury score assays showed that the lung injury level in the AP group was significantly higher than that in the APEExo group (p<0.001; FIG. 7A). Further, the wet/dry weight ratio was significantly higher in the AP group than in the APEExo group (p<0.001; FIG. 7B). In addition, lung function assays revealed significantly lower inspiratory capacity and dynamic compliance in the AP group as compared to the APEExo group, and the resistance was significantly higher in the AP group as compared to the APEExo group (p=0.006, p<0.001, and p<0.001, respectively; FIGS. 7C to 7E).

Moreover, the total and differential cell counts in the BALFs of the AP group were also significantly higher than those of the APEExo group (all p<0.001; FIGS. 7F to 7I).

Data from the above examples confirmed that sepsis induced by lipopolysaccharide and aspiration pneumonia induced by gastric content can cause ARDS and lung injury. Moreover, the present disclosure provides clear evidence to demonstrate the potent therapeutic capacity of the engineered exosomes loaded with hsa-let-7i-5p miRNA, as administration of the engineered exosomes loaded with hsa-let-7i-5p miRNA mitigates ARDS and lung injury induced by sepsis and aspiration pneumonia.

### Example 5. Engineered Exosomes and hpMSC Stem Cells Show Comparable Therapeutic Effects of Lung Diseases

A widely used LPS-induced monomicrobial sepsis model was adopted for the analysis of the therapeutic effects of exosomes on lung diseases. In brief, intraperitoneal injection of gram-negative endotoxin (25 mg/kg, LPS, Escherichia coli 0127: B8, Sigma-Aldrich, USA) was performed as previously reported (Chang, C. Y. et al., Pharmaceuticals. 2020, 13, 280). Adult male wild-type C57B/L6 mice (7 to 8 weeks old, Taiwan National Laboratory Animal Center, Taipei, Taiwan) were employed for the experiments. Regular laboratory chow and water were provided to all mice with free access. Mice were maintained on a 12:12-hour light-dark cycle. Care and handling of the mice were performed following the US National Institutes of Health guidelines.

In this experiment, the mice were randomized into different groups and subjected to various treatments: intraperitoneal injection of normal saline (0.5 mL, the Sham group); normal saline supplemented with hpMSC exosomes (the MExo group); normal saline supplemented with engineered exosomes (the EExo group); LPS alone (the LPS group); LPS plus hpMSC exosomes (the LMExo group); LPS plus engineered exosomes (the LEExo group); LPS plus inhibitor-treated hpMSC exosomes (the LMExoi group); or LPS plus inhibitor-treated engineered exosomes (the LEExoi group).

In the MExo and LMExo groups, two doses of hpMSC exosomes (1×10⁸ particles per mouse) were administered intraperitoneally at 2 h and 26 h after normal saline or LPS injection, respectively. In the EExo and LEExo groups, two doses of engineered exosomes (1×10⁹ particles per mouse) were administered intraperitoneally at 2 h and 26 h after normal saline or LPS injection, respectively. Similarly, in the LMExoi and LEExoi groups, two doses of inhibitor-treated hpMSC exosomes (1×10⁸ particles per mouse) and inhibitor-treated engineered exosomes (1×10⁹ particles per mouse), respectively, were administered intraperitoneally at 2 h and 26 h after LPS injection. The inhibitor-treated exosomes were prepared to further elucidate the role of hsa-let-7i-5p miRNA. Briefly, the miRNA inhibitor is an oligonucleotide having a sequence of 5′-AACAGCACAAACUACUACCUCA-3′ (SEQ ID NO. 1), which acts to inhibit the function of hsa-let-7i-5p miRNA. Electroporation of the miRNA inhibitor into engineered exosomes and MSC exosomes was performed through precipitation, resuspension, transferal, electroporation (150 V/100 μF), removal of free-floating miRNA, and ultracentrifugation. The pellets of miRNA inhibitor-treated engineered exosomes and MSC exosomes were resuspended and stored at −80° C.

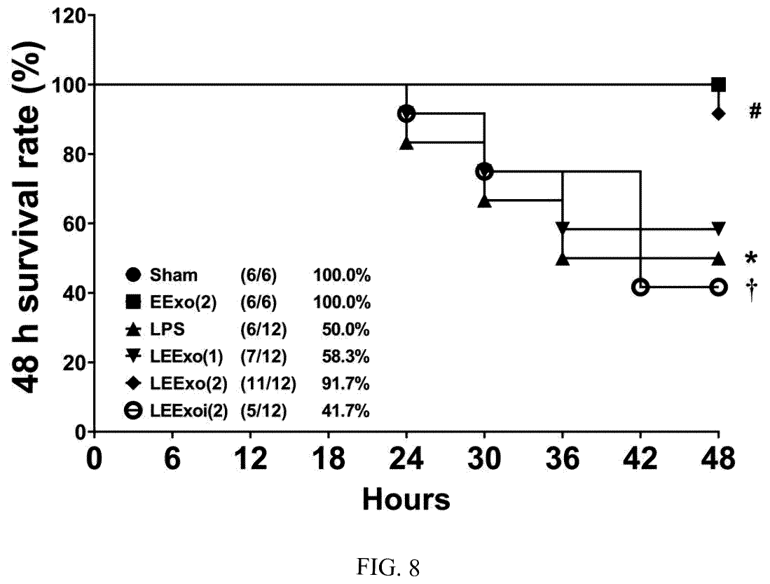

The dosages of MSC exosomes and inhibitor-treated MSC exosomes were determined based on previous data obtained from obese mice complicated with sepsis (Chiang, M. D. et al., Antioxidants. 2022, 11, 615; Chang, C. Y. et al., Pharmaceuticals. 2021, 15, 36). Additionally, the dosage of engineered exosomes was determined based on the preliminary data as shown in FIG. 8), indicating that two doses of 1×10⁹ particles per mouse, but not 1×10⁸ particles per mouse, significantly improved survivorship in LPS-treated mice. Consequently, the dosage of inhibitor-treated engineered exosomes was determined accordingly.

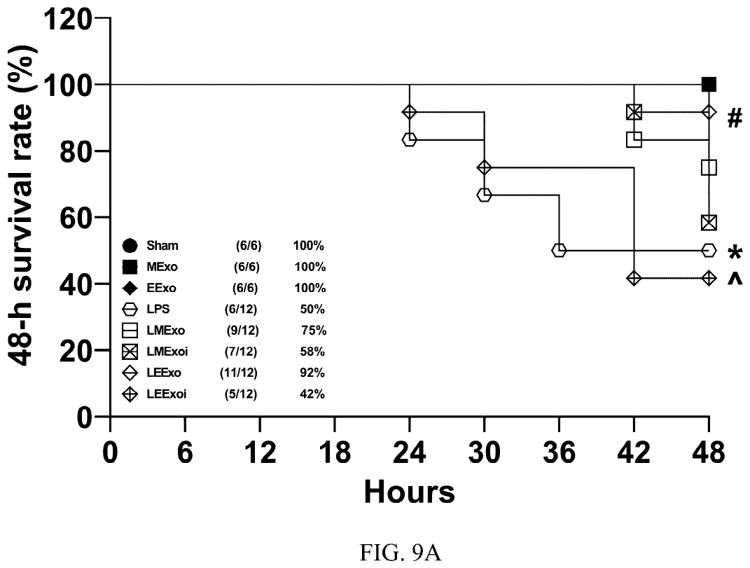

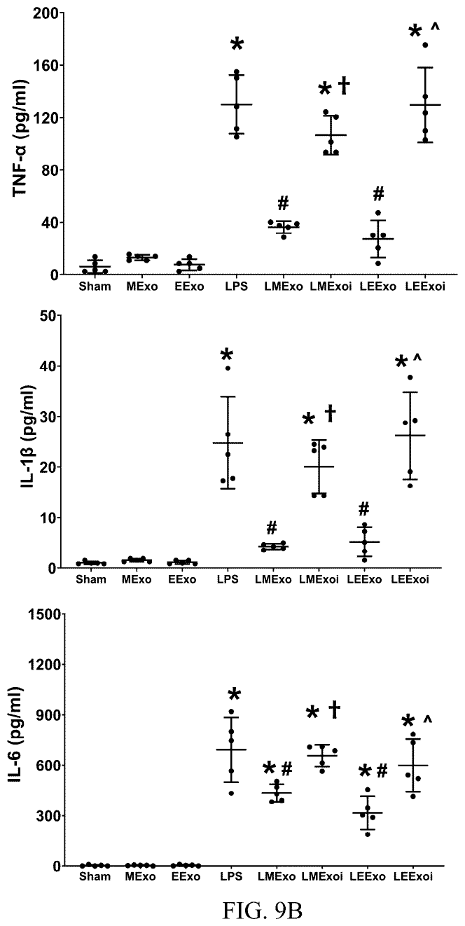

To analyze the survivorship and plasma cytokines of the LPS-induced monomicrobial sepsis animal model, an independent cohort of 78 mice was used. Among them, 18 mice were allocated to the Sham, MExo, and EExo groups (n=6 in each group), and the rest 60 mice were allocated to the LPS, LMExo, LMExoi, LEExo, and LEExoi groups (n=12 in each group). Six mice from each of the Sham, MExo, and EExo groups, along with 12 mice from the LPS, LMExo, LMExoi, LEExo, and LEExoi groups, were utilized. All mice were closely monitored for 48 hours to determine the 48-hour (48-h) survival rate in each group, with the death recognized as blood pressure pulsation disappearance. The survivorship differences between groups were compared using the Kaplan-Meier survivorship plots, as shown in FIG. 9A. The 48-hour survival rates of the Sham, MExo, and EExo groups all reached 100%. In contrast, the LPS group exhibited a significantly lower 48-hour survival rate compared to the Sham group (50% versus 100%, p=0.016), indicating substantial mortality induced by lipopolysaccharide. It was found that the 48-hour survival rate in the LEExo group was significantly higher than that in the LPS group (92% versus 50%, p=0.020), demonstrating the ability of engineered exosomes to mitigate the adverse effects of lipopolysaccharide and enhance survivorship in mice. Conversely, the 48-hour survival rate in the LEExoi group was significantly lower than that in the LEExo group (42% versus 92%, p=0.008), indicating that inhibition of hsa-let-7i-5p significantly diminishes the therapeutic effects of engineered exosomes. A similar trend was observed among the LPS, LMExo, and LMExoi groups. Moreover, the difference between the LMExo and LEExo groups did not reach statistical significance (75% versus 92%, p>0.05), indicating comparable therapeutic effects of MSC exosomes and engineered exosomes.

After the completion of the survivorship determination, five surviving mice from each group were anesthetized, and blood samples were obtained via cardiac puncture to facilitate investigation of systemic inflammation levels. This was assessed by measuring cytokine levels in collected plasma samples. These blood samples were then placed into heparin tubes (Venosafe, Terumo Europe) and centrifuged at 2,000 g for 10 minutes. The resulting supernatant plasma samples were collected and stored at −20° C. for subsequent analysis. The plasma concentrations of cytokines were determined within 7 days after sample collection, using enzyme-linked immunosorbent assay (ELISA). The levels of cytokines, including tumor necrosis factor-α (TNF-α), interleukin-1β (IL-1β), and IL-6, in plasma were assessed using ELISA kits for TNF-α, IL-1β, and IL-6 (all from Enzo Life Science, Farmingdale, NY, USA) and performed as per the manufacturer's protocols.

The plasma cytokines analysis results were shown in FIG. 9B. The TNF-α concentrations in the Sham, MExo, and EExo groups were low and significantly elevated in the LPS group compared to the Sham group (p<0.001). The TNF-α concentrations in the LMExo and LEExo groups were comparable, and both groups showed significantly lower TNF-α concentrations compared to the LPS group (both p<0.001). Conversely, TNF-a concentrations were significantly higher in the LMExoi and LEExoi groups compared to their respective exosome-treated groups (both p<0.001). Similar patterns were observed for IL-1β and IL-6 data. Overall, these results demonstrate that hpMSC exosomes and engineered exosomes achieve similar effects on mitigating lipopolysaccharide-induced systemic inflammation. Moreover, inhibition of hsa-let-7i-5p attenuates the therapeutic effects of both exosome types in this regard.

Then, the lung injury induced by LPS was assessed. An independent cohort of 240 mice was used for lung tissue collection and bronchoalveolar lavage fluid (BALF) collection and analysis. Among them, 54 mice were allocated to the Sham, MExo, and EExo groups (n=18 in each group), 126 mice were allocated to the LPS, LMExoi, and LEExoi groups (n=42 in each group), and 60 mice were allocated to the LMExo and LEExo groups (n=30 in each group). The sample size of each group was determined based on the 48-h survival rate data, to ensure that each group would have at least 18 surviving mice for the following assays.

At 48 hours after LPS or normal saline injection, following euthanasia by decapitation, the lung tissues of the first subset of 6 surviving mice from each group were removed en bloc and promptly snap-frozen in liquid nitrogen, preserving them at −80° C. for subsequent analysis. For the second subset of 6 surviving mice from each group, following euthanasia by decapitation and tracheostomy with tracheostomy tube insertion, the left main bronchus of mice from each group was ligated. Then, the left lung lobes were freshly harvested for wet/dry weight ratio assays to assess lung edema levels. Then, the right lungs infused with 10% formaldehyde (Sigma-Aldrich) under constant pressure for staining purposes in each group were embedded in paraffin wax, serial sectioned, and then stained with hematoxylin and eosin. Morphological characteristics of lung injury were evaluated under a light microscope based on the features of alveolar wall edema, vascular congestion, hemorrhage, and polymorphonuclear (PMN) leukocyte infiltration. Based on the lung injury score scale (0: normal, 5: severe), each histological characteristic was rated, and the sum was calculated to determine lung injury levels. For the wet/dry weight ratio assay (namely, the indicator of tissue water content), the freshly harvested left lung tissues, also from the second set of 6 surviving mice in each group, were weighed and then placed in the oven (80° C., 24 hours) and weighed again when dry. The wet/dry weight ratio was then determined.

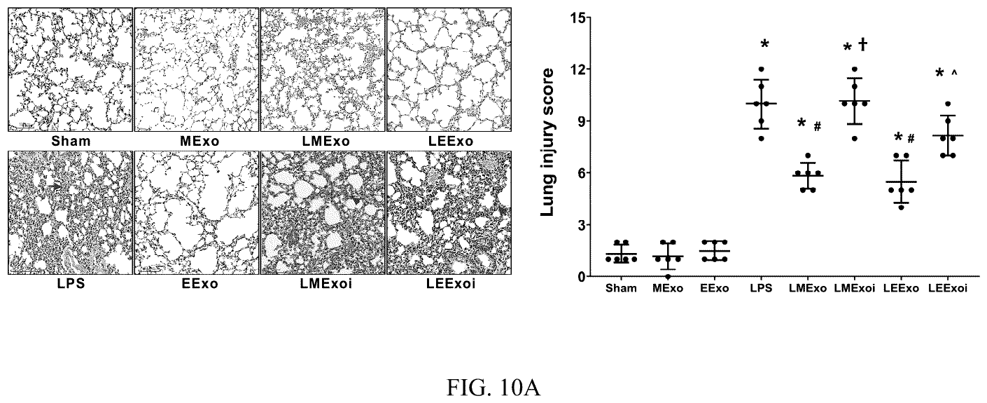

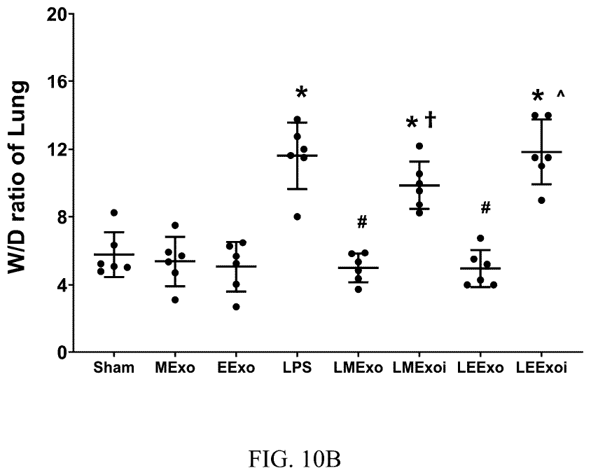

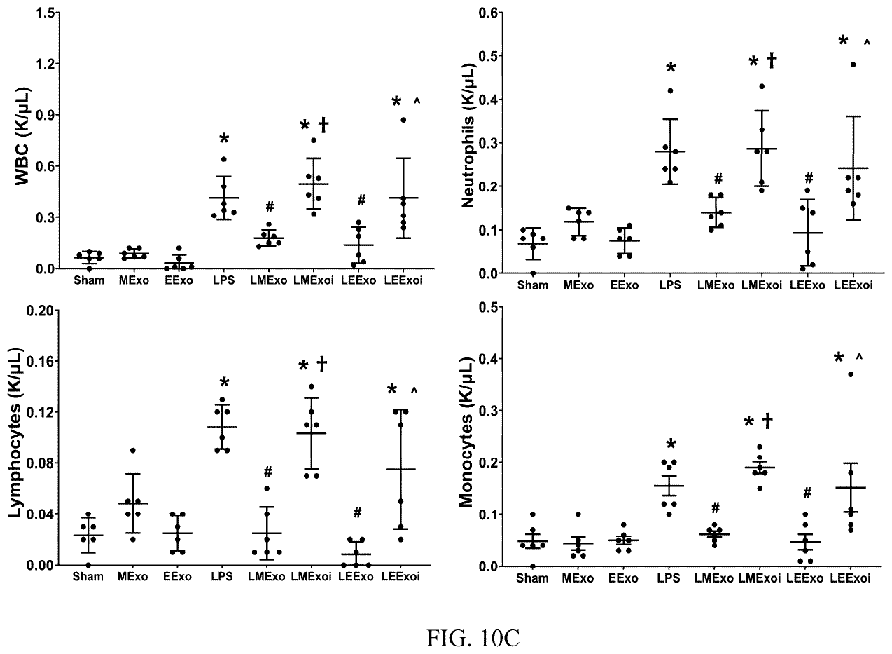

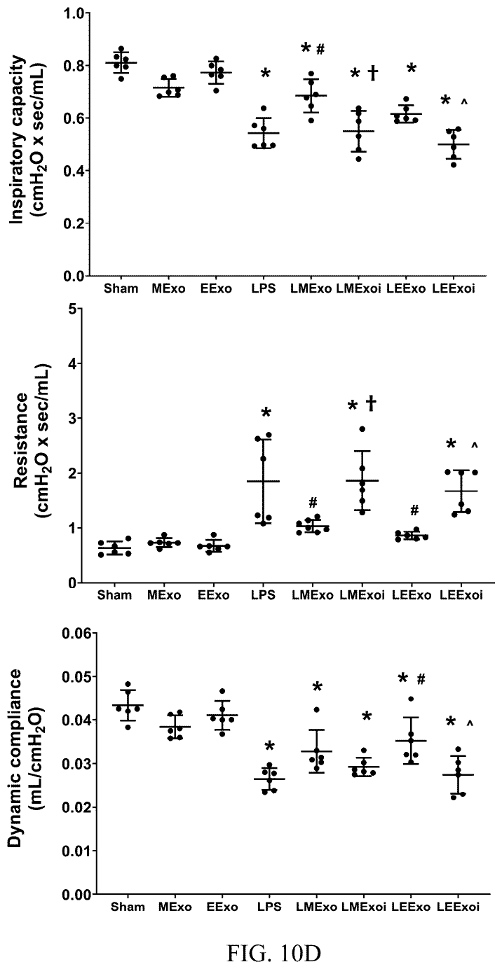

FIG. 10A presents the histological analysis of lung tissues and the corresponding lung injury scores. Histological examination revealed normal lung tissue characteristics in the Sham, MExo, and EExo groups. In contrast, lung tissues from the LPS, LMExo, LMExoi, LEExo, and LEExoi groups exhibited features of lung injury, including increased polymorphonuclear (PMN) infiltration, focal necrosis, and hemorrhages/congestion. Lung injury scores were low in the Sham, MExo, and EExo groups and significantly increased in the LPS group compared to the Sham group (p<0.001). Moreover, lung injury scores in the LMExo and LEExo groups were comparable, and both were significantly reduced compared to the LPS group (both p<0.001). It was found that lung injury scores were significantly higher in the LMExoi and LEExoi groups compared to their respective exosome-treated groups (p<0.001 and p=0.001, respectively). The wet/dry weight ratio result was presented in FIG. 10B.

In addition, the third subset of 6 surviving mice from each group received bronchoalveolar lavage with 1 mL aliquots of sterile normal saline for 5 times, and the bronchoalveolar lavage fluid (BALF) was collected. The total cell counts and differential cell counts were then determined. The BALF data were presented in FIG. 10C.

The data of wet/dry weight ratios of lung tissues (FIG. 10B) and the data of cell counts in BALF (FIG. 10C) both reflect the trends observed in lung injury scores shown in FIG. 10A. These findings collectively underscore the comparable therapeutic efficacy of hpMSC exosomes and engineered exosomes in attenuating lipopolysaccharide-induced lung injury in mice. Additionally, inhibition of hsa-let-7i-5p diminishes the therapeutic effects of both exosome types in this context.

To evaluate lung function, an independent cohort of 80 mice was used. Among them, 18 mice were allocated to the Sham, MExo, and EExo groups (n=6 in each group), 42 mice were allocated to the LPS, LMExoi, and LEExoi groups (n=14 in each group), and 20 mice were allocated to the LMExo and LEExo groups (n=10 in each group). The sample size of each group was determined based on the 48-h survival rate data, to ensure that each group would have at least 6 surviving mice for this assay. An independent cohort of 6 surviving mice from each group was utilized. At 48 hours after LPS or normal saline injection, all 6 surviving mice from each group were anesthetized (zoletil/xylazine, 40/10 mg/kg, i.p.). Subsequently, the mice underwent tracheostomy and insertion of a tracheostomy tube (22 # intravenous catheter; Terumo Corp., Tokyo, Japan) to facilitate pulmonary function assay using a computerized small animal ventilator (flexi Vent FX; SCIREQ Inc., Montreal, Canada). The mechanical ventilation was set at a ventilation rate of 150 breaths/min and a tidal volume of 0.2 mL. Parameters, including inspiratory capacity, airway resistance, and dynamic compliance, were recorded using a flexiWare 8 System (SCIREQ).

As shown in FIG. 10D, inspiratory capacity and dynamic compliance were significantly lower in the LPS group compared to the Sham group (both p<0.001), while airway resistance was significantly higher in the LPS group (p<0.001). In the LMExo group, inspiratory capacity was significantly higher (p=0.001), and airway resistance was significantly lower (p=0.005), as compared to the LPS group. Similarly, the LEExo group showed significantly lower airway resistance (p=0.001) and higher dynamic compliance (p=0.005) compared to the LPS group. Comparing the LMExoi and LMExo groups, inspiratory capacity was significantly lower (p=0.002), and resistance was significantly higher (p=0.004) in the LMExoi group. Similarly, in the LEExoi group compared to the LEExo group, inspiratory capacity and dynamic compliance were significantly lower (p=0.010 and p=0.018, respectively), and resistance was significantly higher (p=0.008). Moreover, the differences in inspiratory capacity, dynamic compliance, and airway resistance between the LMExo and LEExo groups did not reach statistical significance (all p>0.05). Again, these results underscore the comparable therapeutic efficacy of hpMSC exosomes and engineered exosomes in preventing or reversing the lipopolysaccharide-induced lung function loss in mice. Additionally, inhibition of hsa-let-7i-5p diminishes the therapeutic effects of both exosome types in this context.

Also, lung inflammation levels were assessed in the LPS-induced monomicrobial sepsis model, including analyzing activation of upstream regulator nuclear factor-κB (NF-κB) and cytokines in the lungs. The snap-frozen lung tissues from 5 mice, randomly selected from the first set of 6 surviving mice in each group, were used for this assay. Activation of upstream regulator NF-κB in lung tissues was assayed using the Simple Western method. The Simple Western method was conducted as described above. Primary antibodies targeting phosphorylated NF-κB (3033L, Cell Signaling Technology, Danvers, MA, USA) and actin (A2228, Sigma-Aldrich) were employed. The pulmonary concentrations of cytokines were also determined using ELISA. Lung tissue processing was carried out as described in Chiang, M. D. et al., Antioxidants. 2022, 11, 615. The levels of cytokines, including TNF-α, IL-1β, and IL-6, in lung tissues were assessed using ELISA kits for TNF-α, IL-1β, and IL-6 (all from Enzo Life Science, Farmingdale, NY, USA).

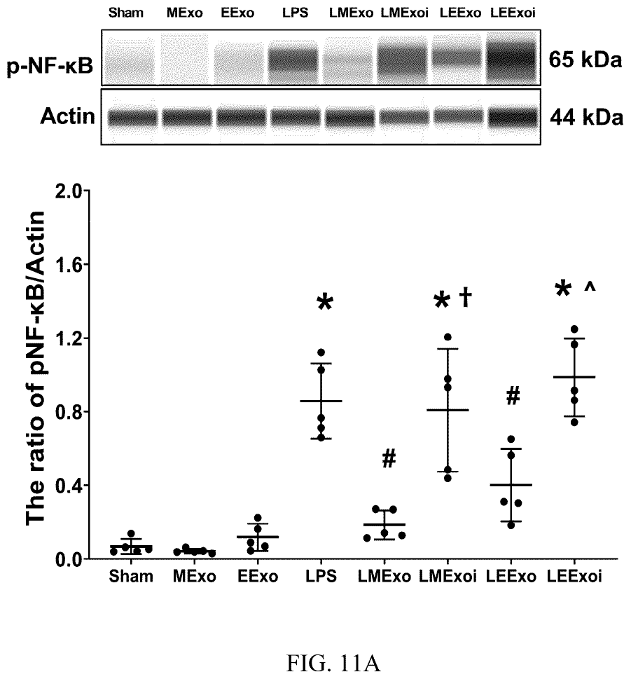

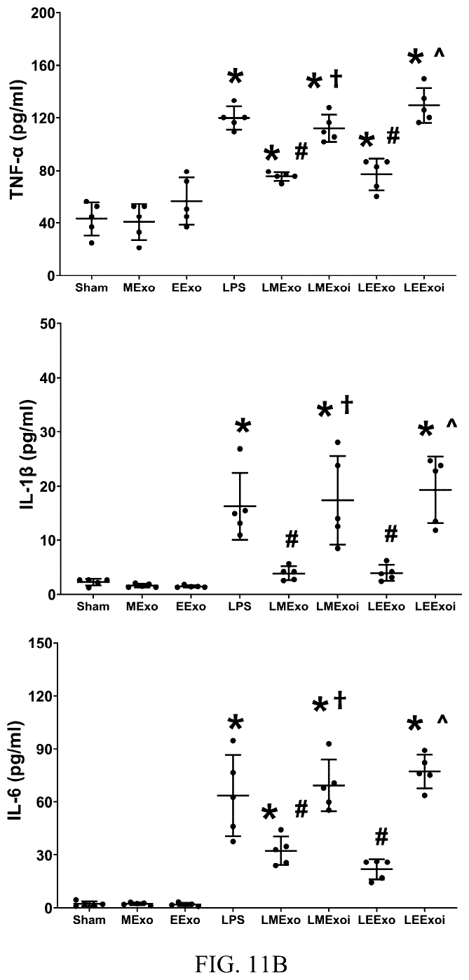

FIG. 11A illustrates the expression levels of the upstream regulator factor NF-κB in lung tissues. Phosphorylated-NF-κB (p-NF-κB) expression levels in the Sham, MExo, and EExo groups were minimal. Conversely, lipopolysaccharide significantly upregulated NF-κB, as evidenced by significantly higher p-NF-κB expression level in the LPS group compared to the Sham group (p<0.001). It was found that expression levels of p-NF-κB in the LMExo and LEExo groups were comparable, and both groups exhibited significantly lower p-NF-κB expression levels compared to the LPS group (p<0.001 and p=0.006, respectively). Moreover, p-NF-κB expression levels were significantly higher in both LMExoi and LEExoi groups compared to their respective exosome-treated groups (both p<0.001). FIG. 11B displays data on TNF-α, IL-1β, and IL-6 expression levels in lung tissues, which mirrored the trends observed in p-NF-κB expression. These findings collectively underscore the comparable efficacy of hpMSC exosomes and engineered exosomes in attenuating lipopolysaccharide-induced lung inflammation in mice. Furthermore, they provide clear evidence of the role of hsa-let-7i-5p in mediating the therapeutic effects of hpMSC exosomes and engineered exosomes in this context.

Furthermore, macrophage polarization in lung tissues of LPS-induced monomicrobial sepsis model was investigated. The snap-frozen lung tissues from 5 mice, randomly selected from the first set of 6 surviving mice in each group, were used for this assay. Activation of hypoxia-inducible factor-1α (HIF-1α, macrophage M1 phase polarization promoter) and inducible nitric oxide synthase (iNOS, macrophage M1 phase polarization marker) in lung tissues was assayed by the Simple Western method. The Simple Western method was conducted as described above. Primary antibodies targeting HIF-1α (IR113-466, iReal Technology), iNOS (IR231-856, iReal Technology), and actin (A2228, Sigma-Aldrich) were used. Expression of iNOS was also assayed using immunohistochemistry staining of iNOS in paraffin sections of lung tissues. The formaldehyde-infused left lung tissues from 5 mice, randomly selected from the second set of 6 surviving mice in each group, were used for this assay. Tissue sections were processed and then incubated with anti-iNOS antibody (IR231-856, iReal Technology), followed by scanning (TissueGnostics Axio Observer Z1 Microscope; TissueGnostics GmbH, Austria) and analysis (Image J, free software provided by NIH, USA).

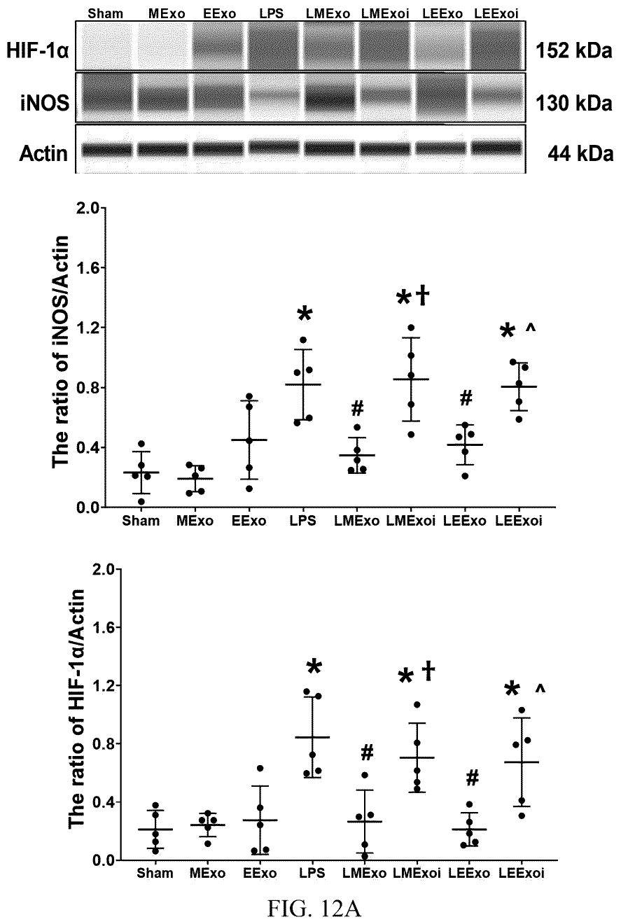

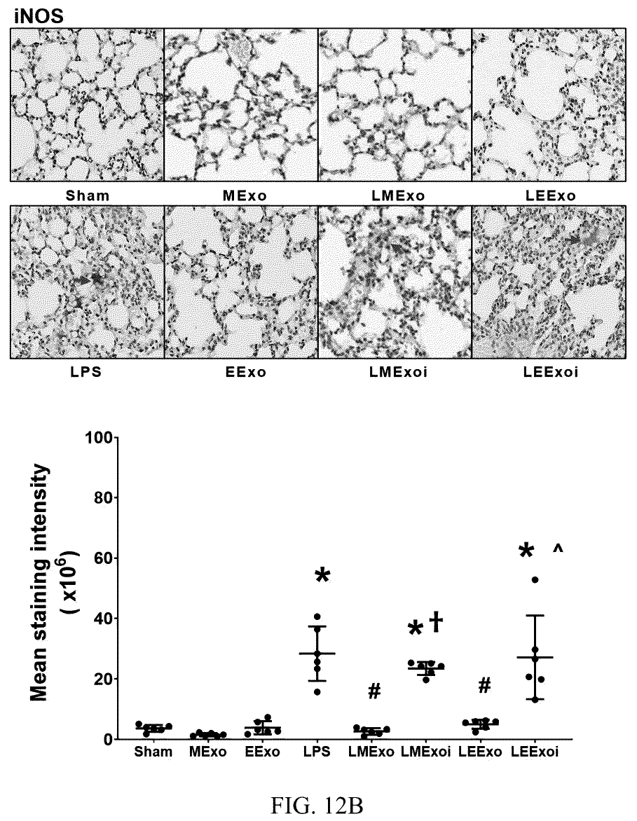

FIG. 12A depicts the expression levels of HIF-1α, the promoter of M1 phase polarization, in lung tissues. Minimal HIF-1α expression was observed in the Sham, MExo, and EExo groups. In contrast, lipopolysaccharide significantly increased HIF-1α expression in the LPS group compared to the Sham group (p=0.001), confirming its effect on upregulating HIF-1α in mouse lung tissues. It was found that HIF-1α expression levels in the LMExo and LEExo groups were comparable, and both groups exhibited significantly lower HIF-1α expression levels compared to the LPS group (p=0.003 and p=0.001, respectively). It was found that the expression levels of HIF-1α were also significantly higher in the LMExoi and LEExoi groups compared to their respective exosome-treated groups (p=0.049 and p=0.032, respectively).

Expression data of the macrophage M1 phase polarization marker, iNOS, presented in FIG. 12A, were obtained from the snap-frozen lung tissues from 5 mice, randomly selected from the 6 surviving mice in each group, as described above. The iNOS expression data presented in FIG. 12B were obtained from the formaldehyde-infused left lung tissues from 5 mice, randomly selected from the 6 surviving mice in each group. Data on the expression levels of iNOS in lung tissues (FIGS. 12A and 12B) mirrored the trends observed in HIF-1α expression (FIG. 12A). These findings collectively highlight the comparable efficacy of hpMSC exosomes and engineered exosomes in attenuating lipopolysaccharide-induced macrophage polarization in mouse lung tissues. Furthermore, they underscore the role of hsa-let-7i-5p in mediating the therapeutic effects of hpMSC exosomes and engineered exosomes in this context.

Further, lung oxidation assays were also carried out, including assay of endogenous antioxidant enzymes and assay of lipid peroxidation. Briefly, the snap-frozen lung tissues from 5 mice, randomly selected from the first set of 6 surviving mice in each group, were used for this assay. Expression of oxidation regulatory enzyme superoxide dismutase 2 (SOD-2) was also measured using the Simple Western method. The Simple Western method was conducted as described above. The primary antibodies targeting SOD-2 (ab68155, Abcam, Cambridge, MA, USA) and actin (A2228, Sigma-Aldrich) were used.

Expression of the marker of oxidative stress was assayed using immunohistochemistry staining of lipid peroxidation-related protein malondialdehyde (MDA, ab27642, Abcam, Cambridge, MA, USA) in paraffin sections of lung tissues. The formaldehyde-infused left lung tissues from 5 mice, randomly selected from the second set of 6 surviving mice in each group, were used for this assay. After processing and incubation with MDA, all tissue sections were observed (TissueGnostics Axio Observer Z1 Microscope, TissueGnostics, Vienna, Austria) and analyzed (Image J).

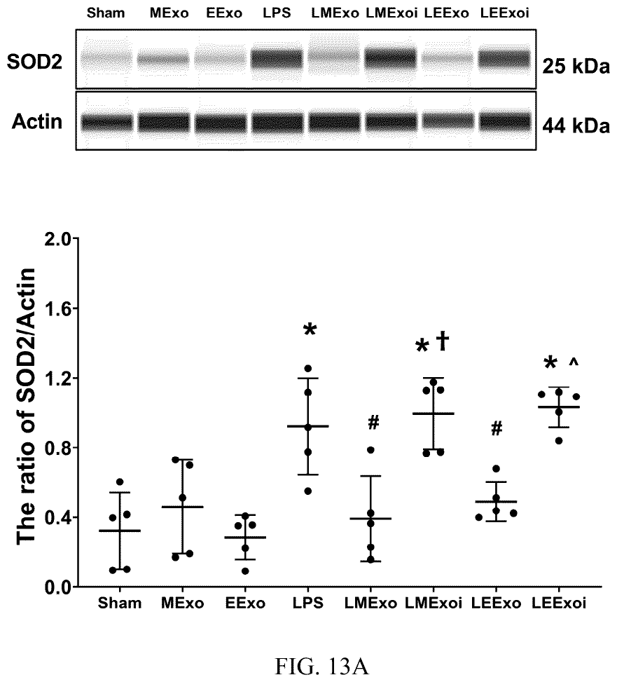

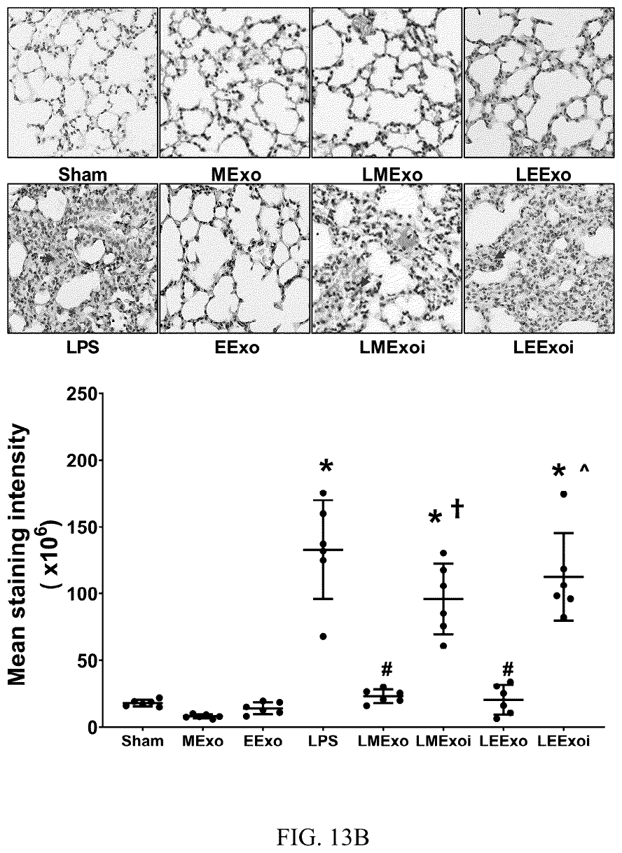

FIG. 13A displays the expression levels of SOD2 in lung tissues. Minimal SOD2 expression was observed in the Sham, MExo, and EExo groups. In contrast, the LPS group exhibited significantly higher SOD2 expression compared to the Sham group (p=0.015). It was found that SOD2 expression levels in the LMExo and LEExo groups were comparable, and both groups showed significantly lower SOD2 expression levels compared to the LPS group (p=0.007 and p=0.043, respectively). Moreover, SOD2 expression was significantly higher in the LMExoi group compared to the LMExo group (p=0.001), and similarly, the LEExoi group exhibited higher SOD2 expression than the LEExo group (p=0.005).

In FIG. 13B, the lipid peroxidation status in lung tissues is illustrated by the levels of MDA (a lipid peroxidation marker). Low MDA levels were observed in the Sham, MExo, and EExo groups. However, the LPS group showed significantly higher MDA levels compared to the Sham group (p<0.001). It was found that MDA levels in the LMExo and LEExo groups were comparable, and both groups exhibited significantly lower MDA levels compared to the LPS group (both p<0.001). Conversely, MDA levels were significantly higher in the LMExoi group compared to the LMExo group (p<0.001), and similarly, the LEExoi group showed higher MDA levels than the LEExo group (p<0.001). These findings collectively demonstrate the comparable effectiveness of hpMSC exosomes and engineered exosomes in mitigating lung oxidation induced by lipopolysaccharide in mice. Additionally, they underscore the role of hsa-let-7i-5p in mediating the therapeutic effects of both exosome types in this context.

Moreover, cell death process and apoptosis in the lungs of LPS-induced monomicrobial sepsis model were also evaluated. The snap-frozen lung tissues from 5 mice, randomly selected from the first set of 6 surviving mice in each group, were used for this assay. Expression of the pro-apoptotic protein cleaved caspase-3 was also assayed using the Simple Western method. The Simple Western method was conducted as described above. The primary antibodies targeting cleaved caspase-3 (IR96-401, iReal Technology) and actin (A2228, Sigma-Aldrich) were employed. On the other hand, apoptosis was measured using the terminal deoxynucleotidyl transferase dUTP nick end labeling (TUNEL) method to detect apoptotic cells in lung tissues using an in-situ cell death detection kit (Roche, USA). The formaldehyde-infused left lung tissues from 5 mice, randomly selected from the second set of 6 surviving mice in each group, were used for this assay. After staining with 4′,6-diamidino-2-phenylindole (DAPI; Pierce) to detect total nuclei, tissue sections were visualized using a confocal microscope (LMS-780), and the TUNEL positive ratio was calculated, as described in Chiang, M. D. et al., Antioxidants. 2022, 11, 615.

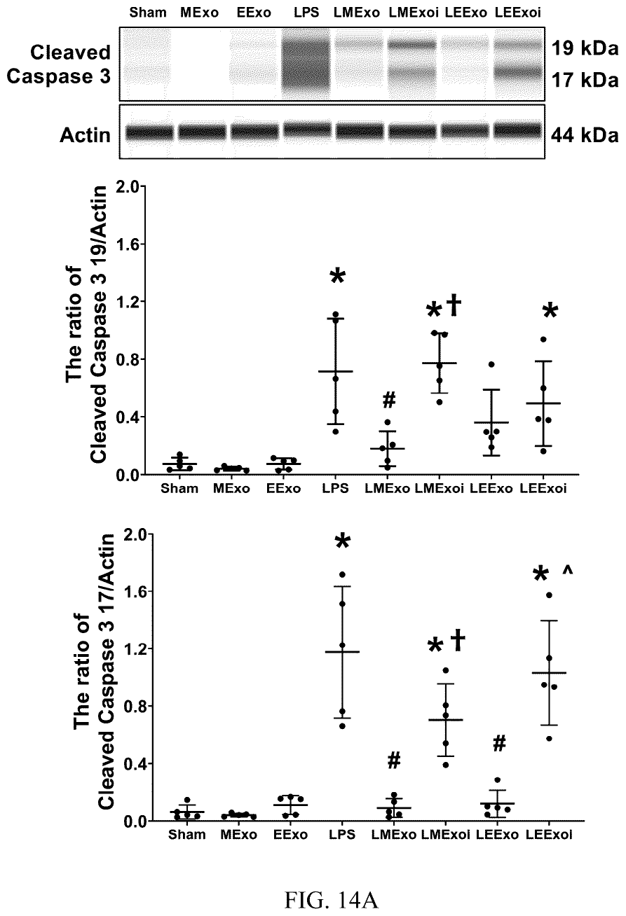

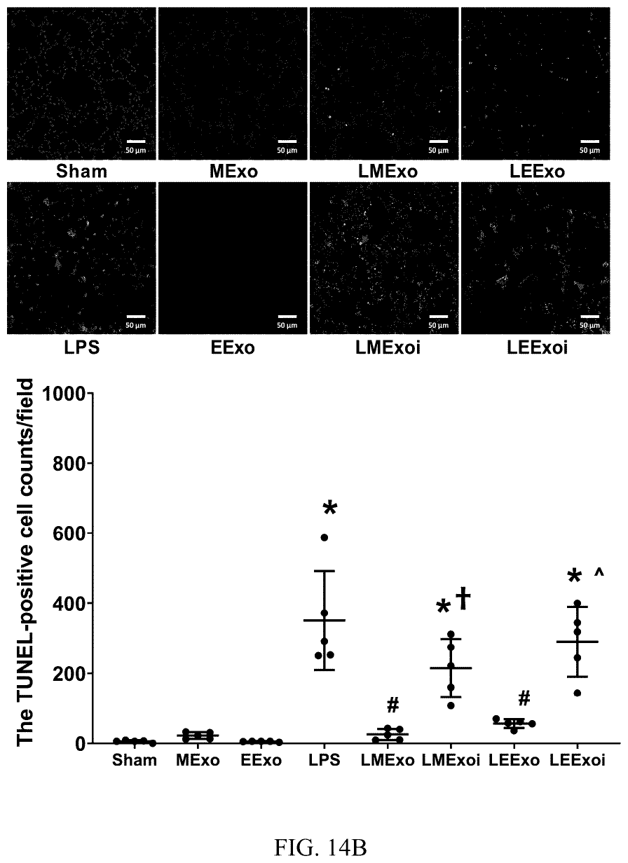

FIG. 14A illustrates the expression levels of the pro-apoptotic protein, cleaved caspase-3 (17 KDa and 19 KDa), in lung tissues. Minimal expression of cleaved caspase-3 (17 KDa) was observed in the Sham, MExo, and EExo groups. Conversely, the LPS group exhibited significantly higher expression levels of cleaved caspase-3 (17 KDa) compared to the Sham group (p<0.001). It was found that expression levels of cleaved caspase-3 (17 KDa) in the LMExo and LEExo groups were comparable, and both groups showed significantly lower cleaved caspase-3 (17 KDa) expression levels compared to the LPS group (both p<0.001). In contrast, the expression levels of cleaved caspase-3 (17 KDa) were also significantly higher in the LMExoi and LEExoi groups compared to their respective exosome-treated groups (p=0.004 and p<0.001, respectively). Data of cleaved caspase-3 (19 KDa) were similar to those of cleaved caspase-3 (17 KDa).

In FIG. 14B, the apoptosis status in lung tissues is illustrated by data on DNA fragmentation measured using the TUNEL assay and the TUNEL-positive cell count. These results mirrored the trends observed in cleaved caspase-3 (17 KDa) expression (FIG. 14A). These findings collectively demonstrate the comparable effectiveness of hpMSC exosomes and engineered exosomes in mitigating lung apoptosis induced by lipopolysaccharide in mice. Additionally, they underscore the role of hsa-let-7i-5p in mediating the therapeutic effects of both exosome types in this context.

While some of the embodiments of the present disclosure have been described in detail in the above, it is, however, possible for those of ordinary skill in the art to make various modifications and changes to the embodiments shown without substantially departing from the teaching of the present disclosure. Such modifications and changes are encompassed in the scope of the present disclosure as set forth in the appended claims.

What is claimed is:

1. A pharmaceutical composition comprising an extracellular vesicle loaded with let-7i-5p miRNA and a pharmaceutically acceptable excipient thereof.

2. The pharmaceutical composition of claim 1, wherein the extracellular vesicle is loaded with let-7i-5p miRNA by electroporation, lipofection, sonication, or contact with calcium chloride.

3. The pharmaceutical composition of claim 2, wherein the extracellular vesicle is derived from an animal cell.

4. The pharmaceutical composition of claim 3, wherein the animal cell is a mammalian cell.

5. The pharmaceutical composition of claim 4, wherein the mammalian cell is a non-stem cell.

6. The pharmaceutical composition of claim 4, wherein the mammalian cell is a mesenchymal stem cell.

7. The pharmaceutical composition of claim 4, wherein the mammalian cell is an immune cell.

8. The pharmaceutical composition of claim 7, wherein the immune cell is a macrophage.

9. The pharmaceutical composition of claim 2, wherein the extracellular vesicle is derived from a cell grown in vitro or a body fluid of a subject.

10. The pharmaceutical composition of claim 1, wherein the extracellular vesicle is an exosome.

11. A method for preventing or treating a lung disease in a subject in need thereof, comprising administering to the subject an effective amount of the pharmaceutical composition of claim 1.

12. The method of claim 11, wherein the lung disease is acute respiratory distress syndrome.

13. The method of claim 12, wherein the acute respiratory distress syndrome is induced by sepsis.

14. The method of claim 12, wherein the acute respiratory distress syndrome is induced by aspiration pneumonia.

15. The method of claim 11, wherein the administration reduces lung injury.

16. The method of claim 15, wherein the administration reduces at least one of inflammation, oxidation, cell death, and apoptosis of lung tissues.

17. The method of claim 11, wherein the administration improves lung function.

18. The method of claim 17, wherein the administration increases at least one of inspiratory capacity, dynamic compliance, and forced expiratory volume of lung.

19. The method of claim 17, wherein the administration lowers at least one of airway resistance and airway elastance of lung.
